(19) United States
(12) Patent Application Publication
(10) Pub. No.: US 2015/0368721 A1
(43) Pub. Date: Dec. 24, 2015

(54) INDEX OF GENOMIC EXPRESSION OF ESTROGEN RECEPTOR (ER) AND ER-RELATED GENES

(71) Applicants: Nuvera Biosciences, Inc., Woburn, MA (US); The Board of Regents of the University of Texas System, Austin, TX (US)

(72) Inventors: W. Fraser SYMMANS, Houston, TX (US); Christos HATZIS, Melrose, MA (US); Lajos PUSZTAI, Bellaire, TX (US)

(21) Appl. No.: 14/601,409

(22) Filed: Jan. 21, 2015

Related U.S. Application Data
(62) Division of application No. 12/772,816, filed on May 3, 2010, now abandoned.
(60) Provisional application No. 61/174,706, filed on May 1, 2009.

(51) Int. Cl. C12Q 1/68; G06F 19/20

(52) U.S. Cl. CPC C12Q 1/6886; G06F 19/20; C12Q 2600/106; C12Q 2600/158

(57) ABSTRACT

The present invention provides the identification and combination of genes that are expressed in tumors that are responsive to a given therapeutic agent and whose combined expression can be used as an index that correlates with responsiveness to that therapeutic agent. One or more of the genes of the present invention may be used as markers (or surrogate markers) to identify tumors that are likely to be successfully treated by that agent or class of agents such as hormonal or endocrine therapy or chemotherapy.

### CROSS-REFERENCE TO RELATED APPLICATIONS

This application is a divisional application of U.S. Non-Provisional patent application Ser. No. 12/772,816 filed May 3, 2010, which claims priority to U.S. Provisional Patent application Ser. No. 61/174,706 filed May 2, 2009, both of which are incorporated herein by reference in their entirety.

### I. FIELD OF THE INVENTION

The present invention relates to the fields of medicine and molecular biology, particularly transcriptional profiling, molecular arrays and predictive tools for response to cancer treatment.

### II. BACKGROUND

Endocrine treatments of breast cancer target the activity of estrogen receptor alpha (ER, gene name ESR1). The current challenges for treatment of patients with ER-positive breast cancer include the ability to predict benefit from endocrine (hormonal) therapy and/or chemotherapy, to select among endocrine agents, and to define the duration and sequence of endocrine treatments. These challenges are each conceptually related to the state of ER activity in a patient's breast cancer. Since ER acts principally at the level of transcriptional control, a genomic index to measure downstream ER-associated gene expression activity in a patient's tumor sample can help quantify ER pathway activity, and thus dependence on estrogen, and intrinsic sensitivity to endocrine therapy. Treatment-specific predictors can enable available multiplex genomic technology to provide a way to specifically address a distinct clinical decision or treatment choice.

### SUMMARY OF THE INVENTION

Embodiments of the invention include methods of calculating an index or score, e.g., an estrogen receptor (ER) reporter index or a sensitivity to endocrine treatment (SET) index, for assessing the hormonal sensitivity of a tumor comprising one or more (each step can be used independently or in combination with other steps) of the steps of: (a) obtaining gene expression data from samples obtained from a plurality of patients; (b) calculating one or more reference gene expression profiles from a plurality of patients with a specific diagnosis, e.g., cancer diagnosis; (c) normalizing the expression data of additional samples to the reference gene expression profile; (d) measuring and reporting estrogen receptor (ER) gene expression from the profile as a method for defining ER status of a cancer; (e) identifying the genes to define a profile to measure ER-related transcriptional activity in any cancer sample; and/or (f) defining one or more reference ER-related gene expression profiles. A “gene profile,” “gene pattern,” “expression pattern” or “expression profile” refers to a specific pattern of gene expression that provides a unique identifier (genes whose expression is indicative of a condition) of a biological sample, for example, a cancer pattern of gene expression, obtained by analyzing a cancer sample and in those cases can be referred to as a “cancer gene profile”. “Gene patterns” can be used to diagnose a disease, make a prognosis, select a therapy, and/or monitor a disease or therapy after comparing the gene pattern to a reference signature. In a further aspect, methods are directed to calculating a weighted index or index (e.g., a sensitivity-to-endocrine-therapy or SET index) based on ER-related gene expression in any patient sample(s) and the ER-related reference profile. In certain aspects methods include combining the measurements of ER gene expression and the index (e.g., weighted index or SET index) for ER-related gene expression to measure and report the gene expression of ER and ER-related transcriptional profile as a continuous or categorical result. In certain aspects the methods assess the likely sensitivity of any cancer to treatment by measuring ER and ER-related gene expression singly or as a combined result and calculating an SET index (a number for comparison purposes) that can be compared to a reference scale to determine the sensitivity of a tumor as it relates to the sensitivity to endocrine treatment. In certain embodiments, the cancer is suspected of being a hormone-sensitive cancer, preferably an estrogen-sensitive cancer. In certain aspects, the suspected estrogen-sensitive cancer is breast cancer. The ER-related genes may include one or more genes selected from a selected set of ER related genes or gene probes. In certain aspects of the invention, ER related genes or gene probes include 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, or 165 ER related genes or gene probes. In particular embodiments one or more genes are selected from Table 2. The weighted or calculated index may be based on similarity with the reference ER-related gene expression profile(s). In certain aspects this similarity is expressed as an index score. In a further aspect of the invention similarity is calculated based on: (a) an algorithm to calculate a distance metric, such as one or a combination of Euclidian, Mahalanobis, or general Miknowski norms; and/or (b) calculation of a correlation coefficient for the sample based on expression levels or ranks of expression levels. The calculation of the weighted or reporter index may include various parameters (e.g., patient covariates) related to the disease condition including, but not limited to the parameters or characteristics of tumor size, nodal status, grade, age, and/or evaluation of prognosis based on distant relapse-free survival (DRFS) or overall survival (OS) of patients.

Embodiments of the invention include patients that are ER-positive and receiving hormonal therapy. In certain aspects the hormonal therapy includes, but is not limited to tamoxifen therapy and may include other known hormonal therapies used to treat cancers, particularly breast cancer. The treatment administered is typically a hormonal therapy, chemotherapy or a combination of the two. Additional aspects of the invention include evaluation of risk stratification of noncancerous cells and may be used to mitigate or prevent future disease. Still further aspects of the invention include normalization by a single digital standard. The method may further comprise normalizing expression data of the one or more samples to the ER-related gene expression profile. The expression data can be normalized to a digital standard. The digital standard can be a gene expression profile from a reference sample.

Further embodiments of the invention include methods of assessing patient sensitivity to treatment comprising one or more steps of: (a) determining expression levels of the ER gene and/or one or more additional ER-related genes; (b) calculating the value of the ER reporter index (e.g., a SET index); (c) assessing or predicting the response to hormonal therapy based on the value of the index; (d) assessing or predicting the response to an administered treatment (e.g., chemotherapy) based on the value of the index, and/or (e) selecting a treatment(s) for a patient based on consideration of the predicted responsiveness to hormonal therapy and/or chemotherapy.

In yet still further embodiments of the invention include a calculated index for predicting response (e.g., a response to treatment) produced by the method comprising the steps of: (a) obtaining gene expression data from samples obtained from a plurality of cancer patients; (b) normalizing the gene expression data; and (c) calculating an index (e.g., a weighted or SET index) based on the ER gene and one or more additional ER-related gene expression levels in the patient sample. In certain aspects the ER-related genes are selected as described supra. Parameters (e.g., patient covariates) used in conjunction with the calculation of the index includes, but is not limited to tumor size, nodal status, grade, age, evaluation of distant relapse-free survival (DRFS) or of overall survival (OS) of the patients and various combinations thereof. Typically, the patients are ER-positive and receiving hormonal therapy, preferably tamoxifen therapy. The methods of the invention may also include treatment administered as a combination of one or more cancer drugs. In particular aspects, the treatment administered is a hormonal therapy, a chemotherapy, or a combination of hormonal therapy and chemotherapy.

In yet still further embodiments of the invention include a calculated index for predicting response to therapy for late-stage (recurrent) cancer as performed by the method comprising the steps of: (a) obtaining gene expression data from samples obtained from a plurality of stage IV cancer patients; (b) normalizing the expression data; (c) calculating an index based on the ER gene and/or one or more additional ER-related gene expression levels in the patient sample; and (d) predicting response to therapy. Typically, the patients are ER-positive and have previously received, or are currently receiving hormonal therapy. The methods of the invention may also include treatment administered as a combination of one or more cancer drugs. In particular aspects, the treatment administered is a hormonal therapy, a chemotherapy, or a combination of hormonal therapy and chemotherapy.

Other embodiments of the invention include methods of assessing, e.g., assessing quantitatively, the estrogen receptor (ER) status of a cancer sample by measuring transcriptional activity comprising two or more of the steps of: (a) obtaining a sample of cancerous tissue from a patient; (b) determining mRNA gene expression levels of the ER gene in the sample; (c) establishing a cut-off ER mRNA value from the distribution of ER transcripts in a plurality of cancer samples, and/or (d) assessing ER status based on the mRNA level of the ER gene in the sample relative to the pre-determined cut-off level of mRNA transcript. The sample may be a biopsy sample, a surgically excised sample, a sample of bodily fluids, a fine needle aspiration biopsy, core needle biopsy, tissue sample, or exfoliative cytology sample. In certain aspects, the patient is a cancer patient, a patient suspected of having hormone-sensitive cancer, a patient suspected of having an estrogen or progesterone sensitive cancer, and/or a patient having or suspected of having breast cancer. In further aspects of the invention, the expression levels of the genes are determined by hybridization, nucleic amplification, or array hybridization, such as nucleic acid array hybridization. In certain aspects the nucleic acid array is a microarray. In still further embodiments, nucleic acid amplification is by polymerase chain reaction (PCR).

Embodiments of the invention may also include kits for the determination of ER status of cancer comprising: (a) reagents for determining expression levels of the ER gene and/or one or more additional ER-related genes in a sample; and/or (b) algorithm and software encoding the algorithm for calculating an ER reporter index from expression of ER and ER-related genes in a sample to determine the sensitivity of a patient to hormonal therapy.

Other embodiments of the invention are discussed throughout this application. Any embodiment discussed with respect to one aspect of the invention applies to other aspects of the invention as well and vice versa. The embodiments in the Example section are understood to be embodiments of the invention that are applicable to all aspects of the invention.

The terms “inhibiting,” “reducing,” or “prevention,” or any variation of these terms, when used in the claims and/or the specification includes any measurable decrease or complete inhibition to achieve a desired result.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specific embodiments presented herein.

FIGS. 1A-1B. Selection of the 165 ER-related reporter genes. (A) Schematic of steps in gene selection. Filtering terms are after normalization and log transformation of expression values: A>5 in p>0.75, retains probe sets with expression level of >5 in at least 75% of the arrays; IQR, inter-quartile range; P95-P5, range between the 95^{th} and 5^{th} percentiles. (B) Selection probabilities P_{g}(50), P_{g}(100), P_{g}(200) for the 200 top-ranking probe sets in terms of their Spearman's rank correlation with the ESR1 transcript (probe set 205225_at) plotted as a function of the probe set's rank in the original dataset. Probabilities were estimated from 1000 bootstrap samples of the original dataset.

FIGS. 2A-2D. Components of the sensitivity to endocrine treatment (SET) in ER-positive and ER-negative cases of the discovery cohort (N=437). Mean expression values of the 59 negatively (X_{N}) and the 106 positively correlated (X_{P}) genes with ESR1 in ER-positive (A) and ER-negative cases (B). Also shown are the raw endocrine index (EI; C) and the scaled and transformed SET index (D) for ER-negative and ER-positive cases as defined by ER gene expression (ESR1 status). All values have been scaled by subtracting the offset of 9.48. For clarity, the SET index as shown in (D) includes the negative values, i.e. was not zero-truncated.

FIGS. 3A-3B. Correlation of SET index classes with DRFS in patients treated with adjuvant tamoxifen in the first validation cohort (n=225 available patients with follow-up data); (A) 8-year follow-up, (B) 16-year follow-up.

FIGS. 4A-4D. Kaplan-Meier estimates of relapse-free survival in patients treated with adjuvant tamoxifen in the second validation cohort, (A) with follow-up censored at 8 years; (B) presented in toto with complete follow up, and presented separately for the subsets with (C) node-negative and (D) node-positive breast cancer. Endocrine sensitivity groups were defined by the SET index. P-values are from the log-rank test.

FIGS. 5A-5B. Correlation of SET index classes with DRFS in patients who did not receive any systemic therapy after surgery in two independent cohorts: (A) Veridex (VDX) cohort, (B) TRANSBIG (TRANS) cohort.

FIGS. 6A-6B. Kaplan-Meier estimates of relapse-free survival in patients with clinically higher risk ER-positive breast cancer who received neoadjuvant chemotherapy (T/FAC) followed by adjuvant endocrine therapy. (A). Endocrine sensitivity groups were defined by the SET index. P-values are from the log-rank test. (B) Contour plot depicting the dependence of the hazard rate of distant relapse or death on residual cancer burden after neoadjuvant chemotherapy (RCB index) and endocrine sensitivity (SET index) according to the Cox regression model of Table 7.

### DETAILED DESCRIPTION OF THE INVENTION

It has already been established that the overall transcriptional profile in breast cancers is dependent on ER status, being largely determined in ER-positive breast cancer by the genomic activity of ER on the transcription of numerous genes (Perou et al., 2000; van't Veer et al., 2002; Gruvberger et al., 2001; Pusztai et al., 2003). The inventors contemplate that the amount of ER-associated reporter gene expression is an indicator of ER transcriptional activity, likely dependence on ER activity, and sensitivity to hormonal therapy. Differences in expression of ER mRNA (the receptor) and ER reporter genes (the transcriptional output) might contribute to variable response of patients with ER-positive breast cancers to hormonal therapy (Buzdar, 2001; Howell and Dowsett, 2004; Hess et al., 2003). Herein, a set of genes are defined that are co-expressed with ER from an independent database of Affymetrix U133A gene profiles from 437 breast cancer subjects and calculated an index score for their expression. Another goal was to determine whether the expression level of ESR1 gene, and value of this index for expression of ER reporter (associated) genes, is associated with distant relapse-free survival (DRFS) in other patients following adjuvant hormonal therapy with tamoxifen.

There are four main approaches to improving the ability to predict responsiveness to cancer therapies. One approach is a standard predictive or chemopredictive study focused on treatment, in which a sufficiently powered discovery population of subjects is used to define a predictive test that must then be proven to be accurate in a similarly sized validation population (Ransohoff, 2005; Ransohoff 2004). Several studies have used this approach to define predictive genes for adjuvant tamoxifen therapy (Ma et al., 2004; Jansen et al., 2005; Loi et al., 2005). There are advantages to this approach, particularly when samples are available from mature studies for retrospective analysis. But two disadvantages are that the study design is empirical and that adjuvant treatment introduces surgery as a confounding variable, because it is impossible to ever know which patients were cured by their surgery and would never relapse, irrespective of their sensitivity to systemic therapy. Neoadjuvant chemotherapy trials enable a direct comparison of tumor characteristics with pathologic response (Ayers et al., 2004). While an empirical study design is needed for chemopredictive studies of cytotoxic chemotherapy regimens because multiple cellular pathways are likely to be disrupted, endocrine therapy of breast cancer specifically targets ER-mediated tumor growth and survival. The compositions and methods of the present invention may define and measure this ER-mediated effect supplanting the need for a limited empirical study design.

A second approach is to identify genes that are downregulated in vivo after treatment with a therapeutic agent. This involves a small sample size of patients who undergo repeat biopsies, but is complicated by the selection of agent and dose used, variable timing of downregulation of different genes after therapy, and variable treatment effect in different tumors.

A third approach is to quantify receptor expression as accurately as possible. Semiquantitative scoring of ER immunoflourescent/immunohistochemical (IFIC) staining is related to disease-free survival following adjuvant tamoxifen (Harvey et al., 1999). For example, measurement of 16 selected genes (mostly related to ER, proliferation, and HER-2) using RT-PCR in a central reference laboratory predicts survival of women with tamoxifen-treated node-negative breast cancer (Paik et al., 2004). In a recent report, measurement of ER mRNA using RT-PCR diagnoses ER IHC status with 93% overall accuracy (Esteva et al., 2005). It was also recently reported that ER mRNA measurements from the same RT-PCR assay predict survival after adjuvant tamoxifen (Paik et al., 2005). So, if gene expression microarrays can reliably measure ER mRNA in a way that can be standardized in different laboratories, those measurements should predict response to endocrine treatment. However, other gene expression measurements from the microarray are informative as well.

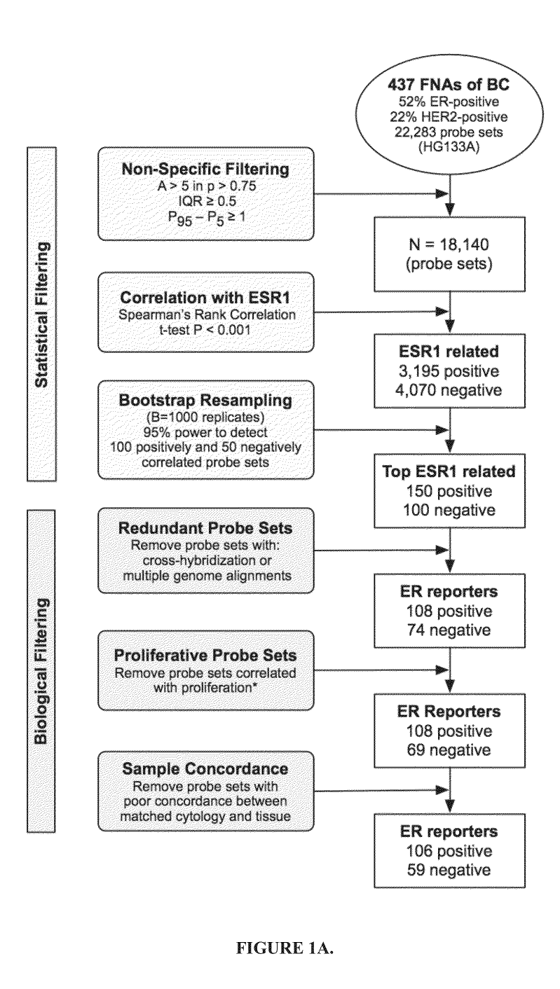

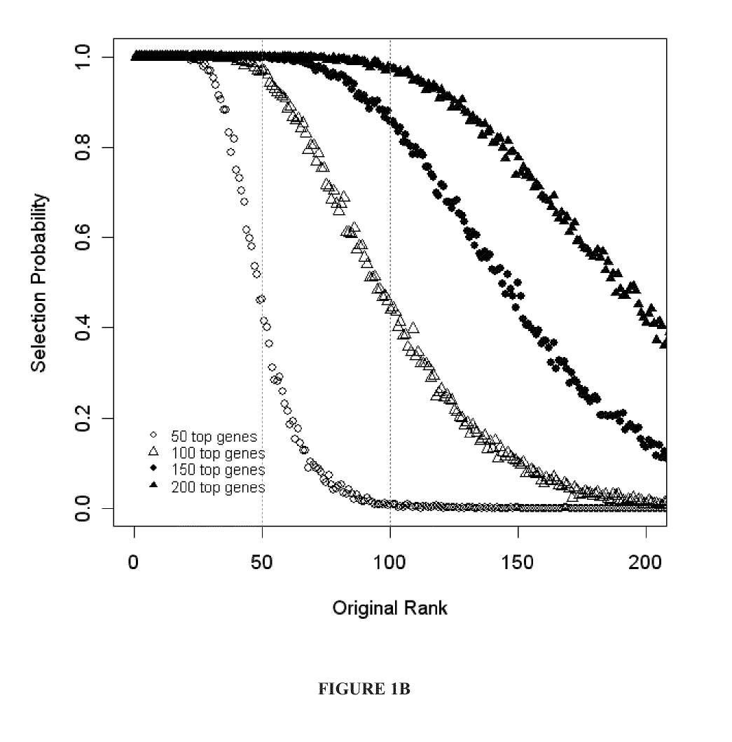

A fourth approach, selected by the inventors, measures the receptor ER gene expression and the transcriptional output from ER activity, taking advantage of the high-throughput microarray platform. This approach theoretically applies to all endocrine treatments and does not require the empirical discovery and validation study populations. If a continuous scale of endocrine responsiveness exists, then specific treatments could be matched to likely response. Some patients would have an excellent response from tamoxifen, but others may need more potent endocrine treatment to respond to the same extent. A challenge with this approach is to accurately define the number and correct ER reporter genes to measure. The approach was to define ER reporter genes from a large, independent data set of 437 breast cancer profiles from Affymetrix U133A arrays. It is not necessary that these patients receive endocrine treatment, or to know their immunohistochemical ER status or survival, in order to define the genes most correlated with ER gene expression. Even with the relatively large sample size of 437 cases, the inventors calculated that 165 genes should be included as reporter genes in order to contain the 50 most ER-related genes with 98.5% confidence and the 100 most related genes with about 90% confidence (FIG. 1). This demonstrates the importance of a sufficiently large reporter gene set to capture a reliable transcriptional signature for ER activity in breast cancers (Perou et al., 2000; Van't Veer et al., 2002; Gruvberger et al., 2001; Pusztai et al., 2003).

If quantitative measurements of the ER-related expression, expression of ER mRNA, and/or ER activity (represented by a calculated index of ER reporter gene expression) accurately predict benefit from therapy, it is possible to develop a continuous genomic scale of measurement for ER expression and activity. This scale could be used to identify subsets of patients with ER-positive breast cancer that: (1) are expected to benefit from tamoxifen alone, (2) require more potent endocrine therapy, (3) may require chemotherapy along with endocrine therapy, or (4) are unlikely to benefit from any combination with endocrine therapy.

To assess expression of at least 5, 25, 50, 100, 150 or 165 reporter (ER-related) genes in a sample, the inventors first developed a gene-expression-based ER associated index. ER-positive and ER-negative reference signatures were then described as the median expression value of each of the 165 reporter genes in the 226 ER-positive and 211 ER-negative subjects, respectively. For new samples, the index is calculated from the mean values of the positive and negative correlated genes with ESR1. If X_{N} and X_{P} are the mean expression value of the 59 negatively-correlated and 106 positively correlated genes with ESR1 in a given sample, then an endocrine reporter index (ERI) is defined as ERI=X_{N}+f (X_{P}−X_{N}), where f is a constant between 0 and 1. Typical values include 0.64, which is the fraction of positively associated genes (106/165) or 0.5. The most typical value is f=0.5. In ER-negative tumors, expression of both the positively and negatively ESR1 correlated genes is low and therefore ERI is small. In ER-positive tumors, expression the positively correlated genes will be greater than that of the negatively correlated genes and therefore the index takes on positive values.

From the ERI, a genomic index of sensitivity to endocrine therapy (SET) was calculated as follows: SET=max {0, A (ERI+B)^{P}_{}}. Constant B is an offset determined to produce positive values for the index, A is an arbitrary scale constant and exponent p was determined through a unconditional Box-Cox power transformation for normality. The most typical values of these constants are A=10, B=−9.48 and p=1.24. The above formulation for SET means that SET is zero-truncated, i.e. if the result of the formula is negative it is set equal to zero.

Embodiments of the present invention also provide a clinically relevant measurement of estrogen receptor (ER) activity within cells by accurately quantifying the transcriptional output due to estrogen receptor activity. This measure or index of the ER pathway or ER activity is an index or measure of the dependence on this growth pathway, and therefore, likely susceptibility to an anti-estrogen receptor hormonal therapy. There are a growing number of hormonal therapies that are used for patients with cancer or to protect from cancer and that vary in their efficacy, cost, and side effects. Aspects of the invention will assist doctors to make improved recommendations about whether and how long to use hormonal therapy for patients with breast cancer or ER-positive breast cancer, particularly those with ER-positive status as established by the existing immunochemical assay, and which hormonal therapy to prescribe for a patient based on the amount of ER-related transcriptional activity measured from a patient's biopsy that indicates the likely sensitivity to hormonal therapy and so matches the treatment selected to the predicted sensitivity to treatment.

Embodiments of the invention are pathway-specific, are applicable to any sample cohort, and are not dependent on inherent biostatistical bias that can limit the accuracy of predictive profiles derived empirically from discovery and validation trial designs linking genes to observed clinical or pathological responses. One advantage of the assay, in addition to its ability to link genomic activity to clinical or pathological response, is that it is quantitative, accurate, and directly comparable using results from different laboratories.

In one aspect of the invention, a calculated index is used to measure the expression of many genes that represent activity of the estrogen receptor pathway within the cells that provides independently predictive information about likely response to hormonal therapy, and that improves the response prediction otherwise obtained by measuring expression of the estrogen receptor alone. The invention includes the methods for standardizing the expression values of future samples to a normalization standard that will allow direct comparison of the results to past samples, such as from a clinical trial. The invention also includes the biostatistical methods to calculate and report the results.

In certain aspects of the invention, measurements of ER and ER-related genes from microarrays have demonstrated to be comparable in standardized datasets from two different laboratories that analyzed two different types of clinical samples (fine needle aspiration cytology samples and surgical tissue samples) and that these accurately diagnose ER status as defined by existing immunochemical assays. In further aspects of the invention, measurements of ER and ER-related genes using this technique have been demonstrated to independently predict distant relapse-free survival in patients who were treated with local therapy (surgery/radiation) followed by post-operative hormonal therapy with tamoxifen. In still further aspects, these gene expression measurements were demonstrated to outperform existing measurements of ER for prediction of survival with this hormonal therapy. In yet still further aspects, measurement of ER-related genes were demonstrated to add to the predictive accuracy of measurements of ER gene expression in the survival analysis of tamoxifen-treated women.

Further embodiments of the invention include kits for the measurement, analysis, and reporting of ER expression and transcriptional output. A kit may include, but is not limited to microarray, quantitative RT-PCR, or other genomic platform reagents and materials, as well as hardware and/or software for performing at least a portion of the methods described. For example, custom microarrays or analysis methods for existing microarrays are contemplated. Also, methods of the invention include methods of accessing and using a reporting system that compares a single result to a scale of clinical trial results. In yet still further aspects of the invention, a digital standard for data normalization is contemplated so that the assay result values from future samples would be able to be directly compared with the assay value results from past samples, such as from specific clinical trials.

The clinical relevance for measurements of ER mRNA and ER related genes from microarrays is also demonstrated herein. Some exemplary advantages to the current composition and methods include, but are not limited to: (1) standardized, quantitative reporting of ER mRNA expression that is comparable in different sample types and laboratories, (2) use of different methods for defining genomic profiles to predict response to adjuvant endocrine treatments, and (3) combining ER-related reporter genes expression to develop a measurable scale or index of estrogen dependence and likely sensitivity to endocrine therapy.

The performance of certain embodiments of a microarray-based ER determination is presented in relation to the current immunohistochemical “gold” standard for evaluation of ER. It is important to remember that IHC assays for ER in routine clinical use are imperfect. The existing IHC assay for ER has only modest positive predictive value (30-60%) for response to various single agent hormonal therapies (Bonneterre et al., 2000; Mouridsen et al., 2001). There are also occasional false negative results. Much of the recognized inter-laboratory differences that affect the IHC results for ER are caused in part by problems associated with tissue fixation methods and antigen retrieval in paraffin tissue sections (Rhodes et al., 2000; Rudiger et al., 2002; Rhodes, 2003; Taylor et al., 1994; Regitnig et al., 2002). Finally, IHC is at least a qualitative assay (reported as positive or negative) and at most a semiquantitative assay (reported as a score). There is still a need to further improve the accuracy with which pathologic assays for ER can predict response to endocrine therapies.

The microarrays provide a suitable method to measure ER expression from clinical samples. ER mRNA levels measured by microarrays, such as Affymetrix U133A gene chips, in fine needle aspirates (FNA), core needle biopsy, and/or frozen tumor tissue samples of breast cancer correlated closely with protein expression by enzyme immunoassay and by routine immunohistochemistry. This is consistent with the previously observed correlation between ER mRNA expression using Northern blot and ER protein expression (Lacroix et al., 2001). An expression level of ER mRNA (ESR1 probe set 205225_)≧500 correctly identified ER-positive tumors (IHC≧10%) with overall accuracy of 96% (95% CI, 90%-99%) in the original set of 82 FNAs and this threshold was validated with 95% overall accuracy (95% CI, 88%-98%) in an independent set of 94 tissue samples (Gong et al. 2007). If any ER staining is considered to be ER-positive, the overall accuracy was 98% for FNAs and 99% for tissues. These results indicate that ER status can be reliably determined from gene expression microarray data, with the advantage of providing comparable results from cytologic and surgical samples, and from different laboratories. With appropriately standardized methods for analysis of data, a microarray platform may also provide robust clinical information of ER status.

ER-positive breast cancer includes a continuum of ER expression that might reflect a continuum of biologic behavior and endocrine sensitivity. Others have reported that some breast cancers are difficult to predict as ER-positive based on transcriptional profile and described non-estrogenic growth effects, such as HER-2, more frequently in this small subset of tumors with aggressive natural history (Kun et al., 2003). Indeed, ER mRNA levels are lower in breast cancers that are positive for both ER and HER2 (Konecny et al., 2003). Another group defined a gene expression signature from cDNA arrays that could predict ER protein levels (enzyme immunoassay) and another signature that predicted flow cytometric S-phase measurements (Gruvberger et al., 2004). Their finding of a reciprocal relationship supports the concept that less ER-positive breast cancers are more proliferative. This relationship is also factored into the calculation of the Recurrence Score that adds the values for proliferation and HER-2 gene groups and subtracts the values for the ER gene group (Paik et al., 2004; Paik et al., 2005). Molecular classification from unsupervised cluster analysis shows the same thing by identifying subtypes of luminal-type (ER-positive) breast cancer (Sorlie et al., 2001). The inverse relationship between ER expression and genes associated with proliferation and other growth pathways is best explained by viewing differentiation as a continuum in which cells become increasingly less proliferative and more dependent on ER stimulation as they differentiate. It follows that there would be an inverse relationship between greater sensitivity to endocrine therapy in differentiated tumors and greater sensitivity to chemotherapy in less differentiated tumors. Measurements along this scale could be valuable for treatment selection.

Randomized clinical trials have demonstrated a survival benefit for some patients who receive additional endocrine therapy with an aromatase inhibitor (compared to placebo) after 5 years of adjuvant tamoxifen (Goss et al., 2003; Bryant and Wolmark, 2003). Although there was a 24% relative reduction in deaths after 2.4 years of letrozole, the absolute difference in recurrence or new primaries was only 2.2% at 2.4 years (Goss et al., 2003, Burnstein, 2003). Without a test to identify patients who actually benefit from prolonged adjuvant endocrine therapy, the resulting decision to provide routine extension of adjuvant endocrine treatment (possibly for an indefinite period) in all women with ER-positive cancer could be a costly and potentially avoidable practice for the healthcare community that would benefit an unidentified minority (Buzdar, 2001). It is therefore helpful to consider that this genomic SET index of ER-associated gene expression might identify patients with intermediate endocrine sensitivity as candidates for extended adjuvant endocrine therapy.

A genomic scale of intrinsic endocrine sensitivity might also provide an improved scientific basis for selection of the most appropriate subjects for inclusion in clinical trials. The ATAC and BIG 1-98 trials enrolled 9,366 and 8,010 postmenopausal women, respectively, and both demonstrated 3% absolute improvement in disease-free survival (DFS) at 5 years from adjuvant aromatase inhibition, compared to tamoxifen (Howell et al., 2005; Thurlimann et al., 2005). Aromatase inhibition as first-line endocrine treatment for all postmenopausal women with ER-positive breast cancer would achieve this survival benefit in 3% of patients at significant cost, and might relegate an effective and less expensive treatment (tamoxifen) to relative obscurity. It is also likely that identification of potentially informative subjects, based on predicted partial endocrine sensitivity from indicators such as the SET index, could reduce the size and cost of adjuvant trials, demonstrate larger absolute survival benefit from improved treatment, and establish who should receive each treatment in routine practice after a positive trial result.

As the cost and complexity of endocrine therapy increase, diagnostic tools are needed not merely for prognosis, but, using strong biological rationale, to demonstrate clinical benefit when they are used to guide the selection and duration of endocrine agents therapy. Indicators such as the SET index can predict response to tamoxifen rather than intrinsic prognosis, and should be independent of stage, grade, and the expression levels of ESR1 and PGR. Continuing validation of the SET index with samples from trials of other hormonal agents would help continual refinement of this clinical interpretation.

In some aspects, although not intending to bound to any single theory, the ER reporter index can be of importance for tumors with high ER mRNA expression. If ER mRNA and the reporter index are high, this can describe a highly endocrine-dependent state for which tamoxifen alone seems to be sufficient for prolonged survival benefit. Patients with high ER mRNA expression but low reporter index appear to derive initial benefit from tamoxifen, but that is not sustained over the long term. Those patients' tumors are likely to be partially endocrine-dependent and might benefit from more potent endocrine therapy in the adjuvant setting. Some women might also benefit from more potent endocrine therapy. A measurable scale of ER gene expression and genomic activity might be applicable to any endocrine therapy that targets ER or other hormonal receptor activity. The relation of an index to efficacy of different endocrine therapies could be used to guide the selection of first-line treatment (e.g., chemotherapy versus endocrine therapy), influence the selection of endocrine agent based on likely endocrine sensitivity, and possibly to re-evaluate endocrine sensitivity if ER-positive breast cancer recurs.

Typically for clinical utility one would define the optimal probe set for ESR1 (ERα gene) on the Affymetrix U133A GeneChip™ to measure ER gene expression. The ESR1 205225_probe set produces the highest median and greatest range of expression and the strongest correlation with ER status because this probe set recognizes the most 3′ end of ESR1 (NetAffx search tool at www.affymetrix.com). The initial reverse transcription (RT) of mRNA sequences in each sample begins at the unique poly-A tail at the 3′ end of mRNA. Therefore, the 3′ end is likely to be the most represented part of any mRNA sequence, and probes that target the 3′ end generally produce the strongest hybridization signal.

In other aspects of the invention it is preferred that biostatistical methods be used that allow standardization of microarray data from any contributing laboratory. At present, direct comparison of IHC results for ER from multiple centers is difficult because technical staining methods differ, positive and negative tissue controls are laboratory-dependent, and interpretation of staining is subjective to the interpretation of the individual pathologist or the threshold setting of the image analysis system being used (Rhodes et al., 2000; Rhodes, 2003; Regitnig et al., 2002). Even in quantitative RT-PCR assays, the expression of genes of interest are calculated relative to only one or several intrinsic housekeeper genes in each assay. The techniques for RNA extraction from fresh samples and preparation for hybridization to Affymetrix microarrays are available from standardized laboratory protocols. However, it should not be overlooked that uniform normalization of microarray data from every breast cancer sample to a digital standard will consistently calculate the expression of all genes of interest relative to the expression of thousands of intrinsic control genes. This availability of multiple controls to standardize expression levels of all genes on the microarray is a robust mathematical control that can explain the comparable results from measurements of ER mRNA expression levels in different sample types and in different laboratories. Adoption of a standard for data normalization of breast cancer samples using the Affymetrix U133A array could lead to a digital standard available to laboratories for clinical trials and for routine diagnostics.

The implications of establishing standard analysis tools for development of a useful clinical assay are clear. When diagnostic microarrays are introduced into the clinic through a central reference laboratory, then uniform data normalization and standardized experimental procedure require internal quality control procedures by the central laboratory. However, in a decentralized system where each center performs its own profiling following a standard procedure using the same microarray platform, a single digital standard should be available for data normalization. This allows different laboratories to generate data that is directly comparable to a common standard.

In addition to other known methods of cancer therapy, hormone therapies may be employed in the treatment of patients identified as having hormone sensitive cancers. Hormones, or other compounds that stimulate or inhibit these pathways, can bind to hormone receptors, blocking a cancer's ability to get the hormones it needs for growth. By altering the hormone supply, hormone therapy can inhibit growth of a tumor or shrink the tumor. Typically, these cancer treatments only work for hormone-sensitive cancers. If a cancer is hormone sensitive, a patient might benefit from hormone therapy as part of cancer treatment. Sensitive to hormones is usually determined by taking a sample of a tumor (biopsy) and conducting analysis in a laboratory.

Cancers that are most likely to be hormone-receptive include: Breast cancer, Prostate cancer, Ovarian cancer, and Endometrial cancer. Not every cancer of these types is hormone-sensitive, however. That is why the cancer must be analyzed to determine if hormone therapy or some combination with chemotherapy is appropriate.

Hormone therapy may be used in combination with other types of cancer treatments, including surgery, radiation and chemotherapy. A hormone therapy can be used before a primary cancer treatment, such as before surgery to remove a tumor. This is called neoadjuvant therapy. Hormone therapy can sometimes shrink a tumor to a more manageable size so that it's easier to remove during surgery.

Hormone therapy is sometimes given in addition to the primary treatment—usually after—in an effort to prevent the cancer from recurring (adjuvant therapy). In some cases of advanced (metastatic) cancers, such as in advanced prostate cancer and advanced breast cancer, hormone therapy is sometimes used as a primary treatment.

Hormone therapy can be given in several forms, including: (A) Surgery—Surgery can reduce the levels of hormones in your body by removing the parts of your body that produce the hormones, including: Testicles (orchiectomy or castration), Ovaries (oophorectomy) in premenopausal women, Adrenal gland (adrenalectomy) in postmenopausal women, Pituitary gland (hypophysectomy) in women. Because certain drugs can duplicate the hormone-suppressive effects of surgery in many situations, drugs are used more often than surgery for hormone therapy. And because removal of the testicles or ovaries will limit an individual's options when it comes to having children, younger people are more likely to choose drugs over surgery. (B) Radiation—Radiation is used to suppress the production of hormones. Just as is true of surgery, it's used most commonly to stop hormone production in the testicles, ovaries, and adrenal and pituitary glands. (C) Pharmaceuticals—Various drugs can alter the production of estrogen and testosterone. These can be taken in pill form or by means of injection. The most common types of drugs for hormone-receptive cancers include: (1) Anti-hormones that block the cancer cell's ability to interact with the hormones that stimulate or support cancer growth. Though these drugs do not reduce the production of hormones, anti-hormones block the ability to use these hormones. Anti-hormones include the anti-estrogens tamoxifen (Nolvadex) and toremifene (Fareston) for breast cancer, and the anti-androgens flutamide (Eulexin) and bicalutamide (Casodex) for prostate cancer. (2) Aromatase inhibitors—Aromatase inhibitors (AIs) target enzymes that produce estrogen in postmenopausal women, thus reducing the amount of estrogen available to fuel tumors. AIs are only used in postmenopausal women because the drugs can't prevent the production of estrogen in women who haven't yet been through menopause. Approved AIs include letrozole (Femara), anastrozole (Arimidex) and exemestane (Aromasin). It has yet to be determined if AIs are helpful for men with cancer. (3) Luteinizing hormone-releasing hormone (LH-RH) agonists and antagonists—LH-RH agonists—sometimes called analogs—and LH-RH antagonists reduce the level of hormones by altering the mechanisms in the brain that tell the body to produce hormones. LH-RH agonists are essentially a chemical alternative to surgery for removal of the ovaries for women, or of the testicles for men. Depending on the cancer type, one might choose this route if they hope to have children in the future and want to avoid surgical castration. In most cases the effects of these drugs are reversible. Examples of LH-RH agonists include: Leuprolide (Lupron, Viadur, Eligard) for prostate cancer, Goserelin (Zoladex) for breast and prostate cancers, Triptorelin (Trelstar) for ovarian and prostate cancers and abarelix (Plenaxis).

One class of pharmaceuticals is the Selective Estrogen Receptor Modulators or SERMs. SERMs block the action of estrogen in the breast and certain other tissues by occupying estrogen receptors inside cells. SERMs include, but are not limited to tamoxifen (the brand name is Nolvadex, generic tamoxifen citrate); Raloxifene (brand name: Evista), and toremifene (brand name: Fareston).

### EXAMPLES

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

### Material and Methods

Needle biopsy samples (fine needle aspirates—FNAs) were analyzed in order to examine genes correlated with the estrogen receptor (ER). The genes were identified by this method using these samples and methods to standardize data were done in order to facilitate calculation of the SET index consistently in different sample types such as biopsies, resected tissue from an excised tumor, and frozen tumor tissue. The evaluation of the SET index was done in frozen tumor tissue for effect of endocrine therapy and in biopsy samples for effect of chemotherapy.

Patients and Samples.

Studies were conducted as follows:

### Assessment of ER-Correlated Genes:

Samples from 437 patients (226 or 52% were ER-positive) from M. D. Anderson Cancer Center (MDACC) taken prior to pre-operative chemotherapy were evaluated to assess correlation of genes with ESR1. These were all pre-treatment fine needle aspiration (FNA) samples of primary breast cancer. Cells from 1-2 passes were collected into a vial with 1 mL of RNAlater™ solution (Asuragen, Austin Tex.) and stored at −80° C. until use.

### Assessment of SET Index in Treated Patients:

First Validation Cohort:

Initial validation of response to hormonal therapy and for establishing cutpoints in the SET index was done with samples of 245 patients from two different institutions (164 from Guy's Hospital, London UK; 81 from Karolinska Institute, Uppsala, Sweden). These patients were uniformly treated with adjuvant tamoxifen for 5 years and their distant relapse-free survival prognosis was evaluated in association with the predicted SET index.

Second Validation Cohort:

An independent cohort of 310 patients from three different institutions (102 from University of Graz, Austria; 109 from Oxford, London, UK; and 99 from Institut Gustav Roussy, France) also treated uniformly with adjuvant tamoxifen for 5 years was studied for validation of the SET index cutpoints and SET groups. All samples from evaluation and validation cohorts were obtained as frozen tumor tissue. This cohort consisted of frozen tumor tissue from patients with ER-positive invasive breast cancer that were profiled at MDACC (N=201) or JBI (N=109) using only Affymetrix U133A gene expression microarrays.

### Assessment of SET Index in Untreated Patients:

Two different untreated cohorts were also studied to determine whether SET index represents the natural history of ER-positive breast cancer in patients who did not receive any prior hormonal therapy. These cohorts consisted of gene expression data from Affymetrix U133A microarrays derived from frozen tumor samples from patients with node-negative ER-positive breast cancer that were profiled at Veridex LLC (Raritan, N.J.) (VDX, N=209) or JBI (TRANS, N=134) (Table 1).

Assessment of SET Index in Patients Treated with Chemotherapy and Endocrine Therapy:

We studied a chemo-endocrine cohort of 131 patients with ER-positive breast cancer and acceptable microarray quality (subset of the discovery cohort) who received uniform neoadjuvant chemotherapy with paclitaxel, fluorouracil, doxorubicin, and cyclophosphamide (T/FAC), of whom 122 (Table 1) subsequently received adjuvant endocrine therapy with tamoxifen (n=40), an aromatase inhibitor (n=53), or both in sequence (n=29).

All patients at MDACC signed an informed consent for voluntary participation to collect samples for research. At other institutions, fresh tissue samples of surgically resected primary breast cancer were frozen in OCT compound and stored at −80° C. Patient characteristics in the various cohorts are listed in Table 1.

TABLE 1
Patient characteristics

| | First Validation Cohort | | | | | | | | Second Validation Cohort | | | |
|---|---|---|---|---|---|---|---|---|---|---|---|---|
| | Treatment | | | | | | | | | | | |
| | Tamoxifen | | | | | | | | Tamoxifen | | | |
| | GUY | | GUY2 | | KI | | Total | | | | IGR | |
| N | 87 | | 77 | | 81 | | 245 | | 102 | | 99 | |
| Platform | Plus2 | | Plus2 | | U133A | | U133A/Plus2 | | U133A | | U133A | |
| Age | | | | | | | | | | | | |
| <=50 | 3 | (3%) | 6 | (8%) | 1 | (1%) | 10 | (4%) | 13 | (13%) | 3 | (3%) |
| >50 | 84 | (97%) | 71 | (92%) | 72 | (89%) | 227 | (93%) | 89 | (87%) | 96 | (97%) |
| Mean (SD) | 63 | (9) | 64 | (9) | 66 | (10) | 64 | (9) | 63 | (11) | 66 | (8) |
| Nodal status | | | | | | | | | | | | |
| Pos | 58 | (67%) | 36 | (47%) | 48 | (59%) | 142 | (58%) | 46 | (45%) | 35 | (35%) |
| Neg | 29 | (33%) | 41 | (53%) | 22 | (27%) | 92 | (38%) | 51 | (50%) | 64 | (65%) |
| NA | — | | — | | 11 | (14%) | 11 | (5%) | 5 | (3%) | — | |
| T stage | | | | | | | | | | | | |
| 1 | 43 | (49%) | 34 | (44%) | 20 | (25%) | 97 | (40%) | 44 | (43%) | 43 | (%43) |
| 2 | 42 | (48%) | 42 | (55%) | 53 | (65%) | 137 | (56%) | 45 | (44%) | 52 | (53%) |
| 3 | 2 | (2%) | 1 | (1%) | — | | 3 | (1%) | 13 | (13%) | 4 | (4%) |
| NA | — | | — | | 8 | (10%) | 8 | (3%) | — | | — | |
| Grade | | | | | | | | | | | | |
| 1 | 17 | (20%) | 14 | (18%) | 12 | (15%) | 43 | (18%) | 21 | (21%) | 24 | (24%) |
| 2 | 48 | (55%) | 34 | (44%) | 42 | (52%) | 124 | (51%) | 59 | (58%) | 52 | (53%) |
| 3 | 16 | (18%) | 24 | (31%) | 14 | (17%) | 54 | (22%) | 20 | (20%) | 23 | (23%) |
| NA | 6 | (7%) | 5 | (7%) | 13 | (16%) | 24 | (10%) | 2 | (1%) | — | |
| AJCC Stage | | | | | | | | | | | | |
| I | 17 | (20%) | 22 | (29%) | 6 | (7%) | 45 | (18%) | 24 | (24%) | 32 | (32%) |
| II | 68 | (78%) | 54 | (70%) | 64 | (79%) | 186 | (76%) | 63 | (62%) | 57 | (58%) |
| III | 2 | (2%) | 1 | (1%) | 0 | | 3 | (1%) | 6 | (6%) | 10 | (10%) |
| NA | — | | — | | 11 | (14%) | 11 | (5%) | 9 | (8%) | — | |
| PR Status | | | | | | | | | | | | |

| | | | | | | | | | | | |
|---|---|---|---|---|---|---|---|---|---|---|---|
| Pos | 64 | (74%) | 59 | (77%) | 71 | (88%) | 194 | (79%) | — | 77 | (78%) |
| Neg | 21 | (24%) | 18 | (23%) | 8 | (10%) | 47 | (19%) | — | 22 | (22%) |

| | | | | | | | | | |
|---|---|---|---|---|---|---|---|---|---|
| NA | 2 | (2%) | — | 2 | (2%) | 4 | (2%) | 102 | — |

| | Second Validation Cohort | | | | Untreated Cohorts | | | | Chemo/Endocrine | |
|---|---|---|---|---|---|---|---|---|---|---|
| | Treatment | | | | | | | | | |
| | Tamoxifen | | | | None | | | | T/FAC, Tam/Al | |
| | OXF | | Total | | VDX | | TRANS | | MDA | |
| N | 109 | | 310 | | 209 | | 134 | | 122 | |
| Platform | U133A | | U133A | | U133A | | U133A | | U133A | |
| Age | | | | | | | | | | |
| <=50 | 15 | (14%) | 31 | (10%) | 90 | (43%) | 95 | (71%) | 61 | (50%) |
| >50 | 94 | (86%) | 279 | (90%) | 119 | (57%) | 39 | (29%) | 61 | (50%) |
| Mean (SD) | 64 | (10) | 64 | (10) | 54 | (12) | 47 | (7) | 52 | (10) |
| Nodal status | | | | | | | | | | |
| Pos | 37 | (34%) | 118 | (38%) | 0 | | 0 | | 80 | (66%) |
| Neg | 66 | (61%) | 181 | (58%) | 209 | | 134 | | 42 | (34%) |
| NA | 6 | (5%) | 11 | (4%) | — | | — | | — | |
| T stage | | | | | | | | | | |
| 1 | 46 | (42%) | 133 | (43%) | 111 | (53%) | 76 | (57%) | 9 | (7%) |
| 2 | 54 | (50%) | 151 | (49%) | 92 | (44%) | 58 | (43%) | 75 | (61%) |
| 3 | 7 | (6%) | 24 | (8%) | 6 | (3%) | 0 | | 20 | (16%) |
| NA | 2 | (2%) | 2 | (1%) | — | | — | | — | |
| Grade | | | | | | | | | | |
| 1 | 21 | (19%) | 66 | (21%) | 4 | (2%) | 29 | (22%) | 12 | (10%) |
| 2 | 51 | (47%) | 162 | (52%) | 36 | (17%) | 69 | (51%) | 75 | (61%) |
| 3 | 17 | (16%) | 60 | (19%) | 102 | (49%) | 36 | (27%) | 35 | (29%) |
| NA | 20 | (18%) | 22 | (7%) | 67 | (32%) | — | | — | |
| AJCC Stage | | | | | | | | | | |
| I | 32 | (29%) | 88 | (28%) | 111 | (53%) | 76 | (57%) | 1 | (1%) |
| II | 63 | (58%) | 183 | (59%) | 92 | (44%) | 58 | (43%) | 78 | (64%) |
| III | 6 | (6%) | 22 | (7%) | 6 | (3%) | 0 | | 43 | (35%) |
| NA | 8 | (7%) | 17 | (5%) | — | | — | | — | |
| PR Status | | | | | | | | | | |

| | | | | | | | |
|---|---|---|---|---|---|---|---|
| Pos | — | 77 | (25%) | — | — | 87 | (71%) |
| Neg | — | 22 | (7%) | — | — | 35 | (29%) |
| NA | 109 | 211 | (68%) | 209 | 134 | | |

Patients in this study had invasive breast carcinoma and were characterized for estrogen receptor (ER) expression using immunohistochemistry (IHC) and/or enzyme immunoassay (EIA). Immunohistochemical (IHC) assay for ER was performed on formalin-fixed paraffin-embedded (FFPE) tissue sections or Camoy's-fixed FNA smears using the following methods: FFPE slides were first deparaffinized, then slides (FFPE or FNA) were passed through decreasing alcohol concentrations, rehydrated, treated with hydrogen peroxide (5 minutes), exposed to antigen retrieval by steaming the slides in tris-EDTA buffer at 95° C. for 45 minutes, cooled to room temperature (RT) for 20 minutes, and incubated with primary mouse monoclonal antibody 6F1 1 (Novacastra/Vector Laboratories, Burlingame, Calif.) at a dilution of 1:50 for 30 minutes at RT (Gong et al., 2004). The Envision method was employed on a Dako Autostainer instrument for the rest of the procedure according to the manufacturer's instructions (Dako Corporation, Carpenteria, Calif.). The slides were then counterstained with hematoxylin, cleared, and mounted. Appropriate negative and positive controls were included. The 96 breast cancers from OXF were ER-positive by enzyme immunoassay as previously described, containing >10 femtomoles of ER/mg protein (Blankenstein et al., 1987).

Estrogen Receptor (ER) Expression was Characterized Using Immunohistochemistry (IHC) and/or Enzyme Immunoassay (EIA).

Breast cancers were defined as ER-positive if nuclear immunostaining was ≧10% tumor cells or Allred score was ≧3, or if enzyme immunoassay identified ≧10 femtomoles ER/mg protein. Low expression (<10%) is reported in routine patient care as negative, but some of those patients potentially benefit from hormonal therapy (Harvey et al., 1999).

RNA Extraction and Gene Expression Profiling.

RNA was extracted from the samples using the RNAeasy Kit™ (Qiagen, Valencia Calif.). The amount and quality of RNA was assessed with DU-640 U.V. Spectrophotometer (Beckman Coulter, Fullerton, Calif.) and it was considered adequate for further analysis if the OD260/280 ratio was ≧1.8 and the total RNA yield was ≧1.0 μg. RNA was extracted from the tissue samples using Trizol (InVitrogen, Carlsbad, Calif.) according to the manufacturer's instructions. The quality of the RNA was assessed based on the RNA profile generated by the Bioanalyzer (Agilent Technologies, Palo Alto, Calif.). Differences in the cellular composition of the FNA and tissue samples have been reported previously (Symmans et al., 2003). In brief, FNA samples on average contain 80% neoplastic cells, 15% leukocytes, and very few (<5%) non-lymphoid stromal cells (endothelial cells, fibroblasts, myofibroblasts, and adipocytes), whereas tissue samples on average contain 50% neoplastic cells, 30% non-lymphoid stromal cells, and 20% leukocytes (Symmans et al., 2003). A standard T7 amplification protocol was used to generate cRNA for hybridization to the microarray. No second round amplification was performed. Briefly, mRNA sequences in the total RNA from each sample were reverse-transcribed with SuperScript II in the presence of T7-(dT)24 primer to produce cDNA. Second-strand cDNA synthesis was performed in the presence of DNA Polymerase I, DNA ligase, and Rnase H. The double-stranded cDNA was blunt-ended using T4 DNA polymerase and purified by phenol/chloroform extraction. Transcription of double-stranded cDNA into cRNA was performed in the presence of biotin-ribonucleotides using the BioArray High Yield RNA transcript labeling kit (Enzo Laboratories). Biotin-labeled cRNA was purified using Qiagen RNAeasy columns (Qiagen Inc.), quantified and fragmented at 94° C. for 35 minutes in the presence of 1× fragmentation buffer. Fragmented cRNA from each sample was hybridized to each U133A gene chip, overnight at 42° C.

Microarray Data Analysis.

The U133A chip contains 22,283 different probe sets that correspond to 13,739 human UniGene clusters (genes). Hybridization cocktail was prepared as described in the Affymetrix technical manual. Raw data generated from Affymetrix chip reader were saved as CEL files. Bioconductor software, which can be found on the World Wide Web at bioconductor.org, was used to generate probe-level intensities and quality measures for each chip. Each chip was normalized using MAS5.0 (mean=600) using the Bioconductor/R software. Log 2-transformed expression values for each probe set were used in subsequent analyses. A reference set of 1322 breast specific (invariant) genes (“housekeeping genes”) and their mean expression intensities were established from a reference breast cancer sample database obtained from MD Anderson Cancer Center. For each test sample, a nonlinear relationship between the intensities of housekeeping genes in the test sample and those of the reference set was determined by fitting a cubic smoothing spline model. This smoothing spline model was then applied to scale the intensities of all probe sets in the array. This normalization scales the probe set intensities in each sample such that the distribution of the housekeeping genes in the test sample matches the distribution in the reference set. All computations are carried out in the software platform R available on the world wide web at r-project.org.

Definition of ER Reporter Genes.

ER “reporter genes” were defined from a dataset of Affymetrix U133A transcriptional profiles from 437 breast cancer patient samples from the MD Anderson Cancer Center tumor database. Expression data had been normalized to an average probe set intensity of 600 per array using MAS5.0 and then scaled as described above. Expression values were log 2-transformed. The dataset was filtered to include 18140 probe sets with most variable expression, where P₀≧5 in at least 75% of the arrays, P₇₅−P₂₅≧0.5, and P₉₅−P₅≧1 (P_{q} is the q^{th} percentile of log 2-intensity for each probe set). Those were ranked by Spearman's rho (Kendall and Gibbons, 1990) with ER mRNA (ESR1 probe set 205225_at) expression, both positive and negative correlation, of which 3195 probe sets had a significant positive correlation and 4070 a significant negative correlation with ESR1 (t-test of correlation coefficients with one-sided significance level of 99.9%). The size of the reporter gene set was then determined by a bootstrap-based method that accounts for sampling variability in the correlation coefficient and in the resulting probe sets rankings (Pepe et al., 2003). The entire dataset was re-sampled 1000 times with replacement at the subject level (i.e., when one of the 437 subjects was selected in the bootstrap sample, all candidate probe sets from that subject were included in the dataset). Each probe set was ranked according to its correlation with ESR1 in each bootstrap dataset. The probability (P) of selection for each probe set (g) in a reporter gene set of defined length (k) was calculated as P[Rank(g)≦k]. A similar computation provided estimates of the power to detect the truly co-expressed genes from a study of a given size (Pepe et al., 2003).

FIG. 1A describes the process used to select the probe sets (genes) for the SET signature. First, statistical filtering criteria were applied. Minimum intensity and minimum variance criteria were applied to filter out probe sets that did not show enough variation across arrays in the discovery dataset or probe sets that were expressed at low levels. This step eliminated 19% of the probe sets. Then, probe sets were filtered for significant correlation with ESR1 (separately for positive and negative correlations) based on one-sided t-test on Spearman's rank correlation coefficient (one-sided α=0.001). This step eliminated 60% of the probe sets. Finally a bootstrap resampling approach (Pepe et al, 2003) was used to account for sampling variability in the estimation of the correlation coefficients and thus in the rankings of the probe sets to help determine the size of the signatures. Further redundancies were removed based on biological criteria. First, each probe was evaluated in terms of hybridization specificity (cross-hybridizing transcripts) as well as for multiplicity of alignments of the consensus sequence to the genome. Probe annotations were obtained through batch queries on the Affymetrix's public NetAffx analysis center (on the www at affymetrix.com/analysis/index.affx) based on the March 2006 genome assembly (NCBI Build 36.1). Sixty-eight probes that cross-hybridized to multiple mRNA transcripts or mapped to multiple genomic locations were selectively eliminated. Next, to reduce dependency of the index to proliferation effects, five ESR1-negatively correlated probe sets that were positively correlated with genomic grade index (Sotiriou et al, 2006) were eliminated (Spearman's rank correlation >0.5). Finally, we removed twelve probe sets that showed considerable bias between matched cytology and tissue samples from 38 breast cancers (unrelated to the study cohorts). All filtering steps were non-specific, i.e. outcome information was not used in any of the above decisions.

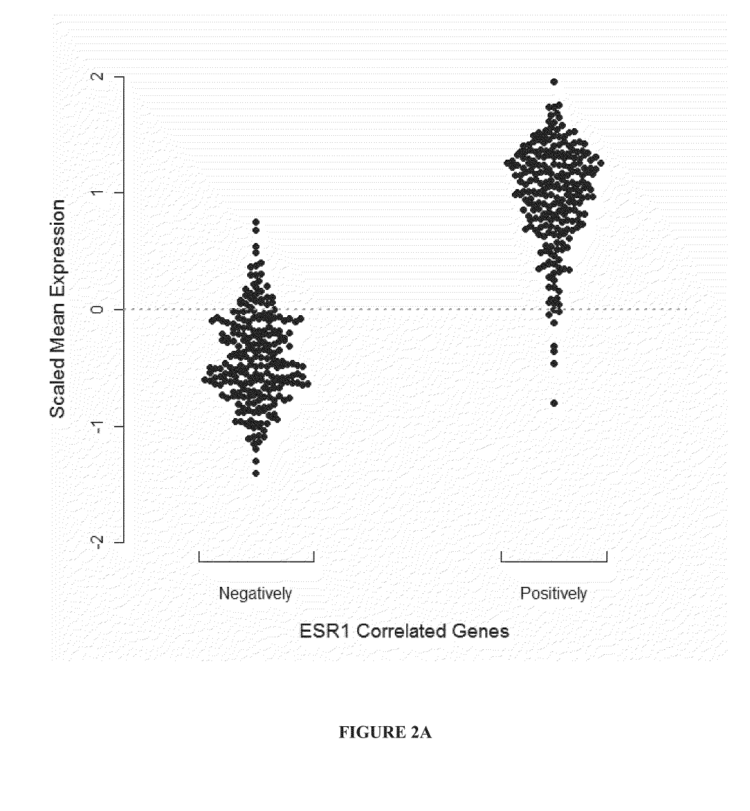

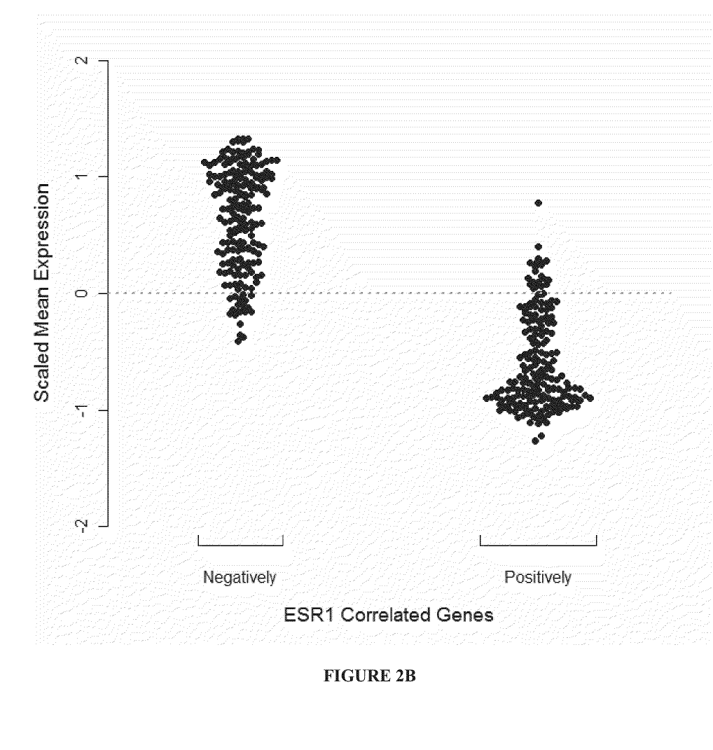

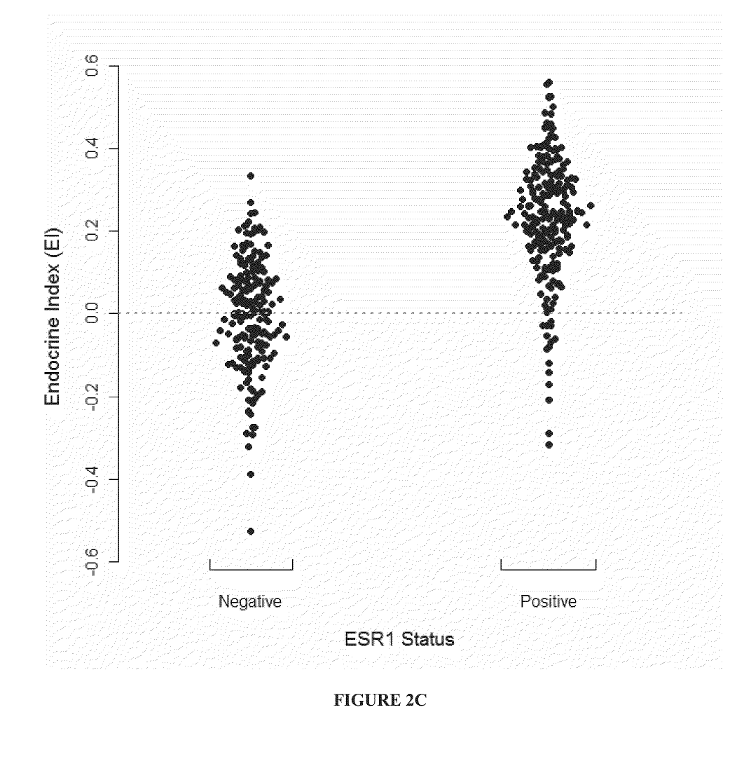

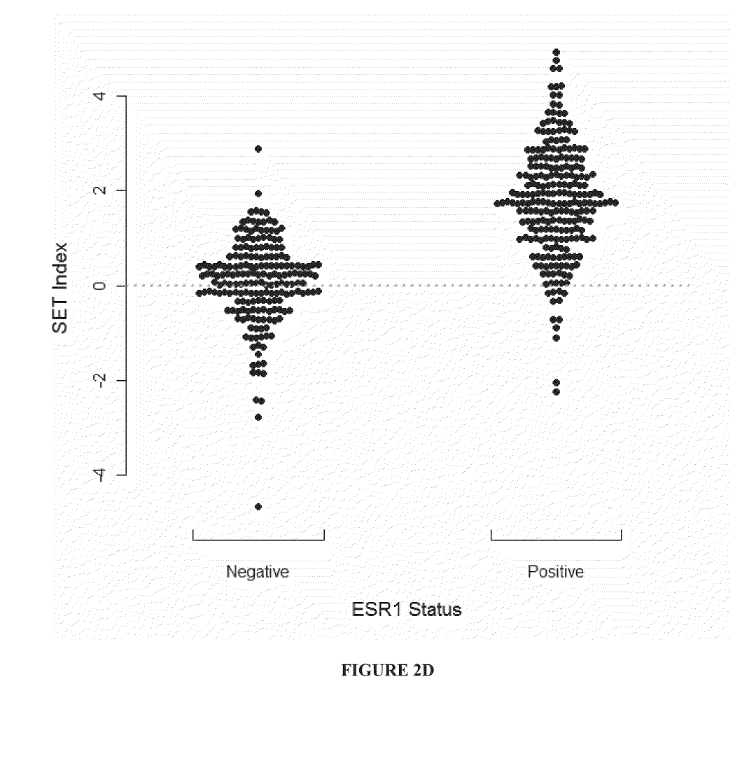

Genes that are truly co-expressed with ESR1 have selection probabilities close to 1, but the selection probability diminishes quickly for lower order probe sets (FIG. 1B). The probability of selecting the top 50 ER-associated probes would be 100% if the ER reporter gene list included 150 probes, 97.1% if 100 probes, and 46.2% if 50 probes (FIG. 1B). An ER reporter list with 200 top-ranking probes would include the top 100 probes with 97.4% probability and the top 150 probes with about 77.7% probability (FIG. 1B). The SET index signature consists of two sets of genes, those that are positively correlated and those that are negatively correlated with ESR1 expression. The following figures show the mean expression values of the ESR1 positively and negatively correlated genes in ER-positive and ER-negative cases from the discovery cohort, as defined by ER gene expression (ESR1 status). As shown, the positively correlated genes are on average expressed more highly in ER-positive disease and the reverse is true for the negatively correlated genes (FIGS. 2A, 2B). As a result, the SET index, which is a combination of the average expression levels of these two groups of genes, is higher in ER-positive disease (FIGS. 2C, 2D).

Table 2 shows all the genes identified to be highly correlated with the estrogen receptor expression. These genes provide robustness to the signature for consistency of performance between expected sample types and for the heterogeneity expected in the ER-positive tumors in terms of recurrence events and other pathologic factors. The genes in Table 2 have been ranked based on strength of correlation to ER expression and have been separately listed based on whether the correlation is negative or positive with respect to ER expression. Table 3 shows the breakdown of samples and data used in the analyses based on available clinical and outcomes data, quality of samples, and acceptable performance of microarrays.

TABLE 2
Genes for ER-related genomic activity, either positively or negatively, and used in calculating index.

| | | | | | |
|---|---|---|---|---|---|
| | | | Entrez | | |
| Probe Set ID | Gene Symbol | Gene Title | Gene ID | Chromosome | Cytoband |

Positive correlation with ESR1

| | | | | | |
|---|---|---|---|---|---|
| 209460_at | ABAT | 4-aminobutyrate aminotransferase | 18 | chr16 | 16p13.2 |
| 205355_at | ACADSB | acyl-Coenzyme A dehydrogenase, short/branched | 36 | chr10 | 10q26.13 |
| | | chain | | | |
| 213245_at | ADCY1 | adenylate cyclase 1 (brain) | 107 | chr7 | 7p13-p12 |
| 204497_at | ADCY9 | adenylate cyclase 9 | 115 | chr16 | 16p13.3 |
| 209173_at | AGR2 | anterior gradient homolog 2 (Xenopus laevis) | 10551 | chr7 | 7p21.3 |
| 211712_s_at | ANXA9 | annexin A9 | 8416 | chr1 | 1q21 |
| 212985_at | APBB2 | amyloid beta (A4) precursor protein-binding, family B, member 2 | 323 | chr4 | 4p14-p13 |
| 40148_at | APBB2 | amyloid beta (A4) precursor protein-binding, family B, member 2 | 323 | chr4 | 4p14-p13 |
| 202641_at | ARL3 | ADP-ribosylation factor-like 3 | 403 | chr10 | 10q23.3 |
| 40093_at | BCAM | basal cell adhesion molecule (Lutheran blood group) | 4059 | chr9 | 19q13.2 |
| 201170_s_at | BHLHE40 | basic helix-loop-helix family, member e40 | 8553 | chr3 | 3p26 |
| 211939_x_at | BTF3 | basic transcription factor 3 | 689 | chr5 | 5q13.2 |
| 203571_s_at | C10orf116 | chromosome 10 open reading frame 116 | 10974 | chr10 | 10q23.2 |
| 221823_at | C5orf30 | chromosome 5 open reading frame 30 | 90355 | chr5 | 5q21.1 |
| 218195_at | C6orf211 | chromosome 6 open reading frame 211 | 79624 | chr6 | 6q25.1 |
| 220581_at | C6orf97 | chromosome 6 open reading frame 97 | 80129 | chr6 | 6q25.1 |
| 203963_at | CA12 | carbonic anhydrase XII | 771 | chr15 | 15q22 |
| 204811_s_at | CACNA2D2 | calcium channel, voltage-dependent, alpha 2/delta subunit 2 | 9254 | chr3 | 3p21.3 |
| 41660_at | CELSR1 | cadherin, EGF LAG seven-pass G-type receptor 1 | 9620 | chr22 | 22q13.3 |
| | | (flamingo homolog, Drosophila) | | | |
| 200810_s_at | CIRBP | cold inducible RNA binding protein | 1153 | chr19 | 19p13.3 |
| 219414_at | CLSTN2 | calsyntenin 2 | 64084 | chr3 | 3q23-q24 |
| 201754_at | COX6C | cytochrome c oxidase subunit VIc | 1345 | chr8 | 8q22-q23 |
| 205081_at | CRIP1 | cysteine-rich protein 1 (intestinal) | 1396 | chr14 | 14q32.33 |
| 219913_s_at | CRNKL1 | crooked neck pre-mRNA splicing factor-like 1 | 51340 | chr20 | 20p11.2 |
| | | (Drosophila) | | | |
| 202263_at | CYB5R1 | cytochrome b5 reductase 1 | 51706 | chr1 | 1p36.13-q41 |
| 206754_s_at | CYP2B6 /// | cytochrome P450, family 2, subfamily B, polypeptide | 1555 /// 1556 | chr19 | 19q13.2 |
| | CYP2B7P1 | 6 /// cytochrome P450, family 2, subfamily B, | | | |
| | | polypeptide 7 pseudogene 1 | | | |
| 210272_at | CYP2B7P1 | cytochrome P450, family 2, subfamily B, polypeptide 7 | 1556 | chr19 | 19q13.2 |
| | | pseudogene 1 | | | |
| 205471_s_at | DACH1 | dachshund homolog 1 (Drosophila) | 1602 | chr13 | 13q22 |
| | DBNDD2 /// | dysbindin (dystrobrevin binding protein 1) domain | | | |
| | SYS1- | containing 2 /// SYS1-DBNDD2 readthrough | 55861 /// | chr20 | |
| 218094_s_at | DBNDD2 | transcript | 767557 | | 20q13.12 |
| 218976_at | DNAJC12 | DnaJ (Hsp40) homolog, subfamily C, member 12 | 56521 | chr10 | 10q22.1 |
| 205066_s_at | ENPP1 | ectonucleotide pyrophosphatase/phosphodiesterase 1 | 5167 | chr6 | 6q22-q23 |
| 214053_at | ERBB4 | v-erb-a erythroblastic leukemia viral oncogene | 2066 | chr2 | 2q33.3-q34 |
| | | homolog 4 (avian) | | | |
| 217838_s_at | EVL | Enah/Vasp-like | 51466 | chr14 | 14q32.2 |
| 218532_s_at | FAM134B | family with sequence similarity 134, member B | 54463 | chr5 | 5p15.2l |
| 213304_at | FAM179B | family with sequence similarity 179, member B | 23116 | chr14 | 14q21.3 |
| 209696_at | FBP1 | fructose-1,6-bisphosphatase 1 | 2203 | chr9 | 9q22.3 |
| 204667_at | FOXA1 | forkhead box A1 | 3169 | chr14 | 14q12-q13 |
| 44654_at | G6PC3 | glucose 6 phosphatase, catalytic, 3 | 92579 | chr17 | 17q21.31 |
| 205354_at | GAMT | guanidinoacetate N-methyltransferase | 2593 | chr19 | 19p13.3 |
| 209603_at | GATA3 | GATA binding protein 3 | 2625 | chr10 | 10p15 |
| 205696_s_at | GFRA1 | GDNF family receptor alpha 1 | 2674 | chr10 | 10q26 |
| 218692_at | GOLSYN | Golgi-localized protein | 55638 | chr8 | 8q23.2 |
| 205862_at | GREB1 | GREB1 protein | 9687 | chr2 | 2p25.1 |
| 201413_at | HSD17B4 | hydroxysteroid (17-beta) dehydrogenase 4 | 3295 | chr5 | 5q21 |
| 203628_at | IGF1R | insulin-like growth factor 1 receptor | 3480 | chr15 | 15q26.3 |
| 204863_s_at | IL6ST | interleukin 6 signal transducer (gp130, oncostatin | 3572 | chr5 | 5q11 |
| | | M receptor) | | | |
| 204686_at | IRS1 | insulin receptor substrate 1 | 3667 | chr2 | 2q36 |
| 203710_at | ITPR1 | inositol 1,4,5-triphosphate receptor, type 1 | 3708 | chr3 | 3p26-p25 |
| 212496_s_at | JMJD2B | jumonji domain containing 2B | 23030 | chr19 | 19p13.3 |
| 217894_at | KCTD3 | potassium channel tetramerisation domain containing 3 | 51133 | chr1 | 1q41 |
| 203144_s_at | KIAA0040 | KIAA0040 | 9674 | chr1 | 1q24-q25 |
| 212441_at | KIAA0232 | KIAA0232 | 9778 | chr4 | 4p16.1 |
| 221874_at | KIAA1324 | KIAA1324 | 57535 | chr1 | 1p13.3 |
| 213234_at | KIAA1467 | KIAA1467 | 57613 | chr12 | 12p13.1 |
| 212442_s_at | LASS6 | LAG1 homolog, ceramide synthase 6 | 253782 | chr2 | 2q24.3 |
| 212692_s_at | LRBA | LPS-responsive vesicle trafficking, beach | 987 | chr4 | 4q31.3 |
| | | and anchor containing | | | |
| 211596_s_at | LRIG1 | leucine-rich repeats and immunoglobulin-like | 26018 | chr3 | 3p14 |
| | | domains 1 | | | |
| 208682_s_at | MAGED2 | melanoma antigen family D, 2 | 10916 | chrX | Xp11.2 |
| 203929_s_at | MAPT | microtubule-associated protein tau | 4137 | chr17 | 17q21.1 |
| 209623_at | MCCC2 | methylcrotonoyl-Coenzyme A carboxylase 2 (beta) | 64087 | chr5 | 5q12-q13 |
| 214077_x_at | MEIS3P1 | Meis homeobox 3 pseudogene 1 | 4213 | chr19 | 17p12 |
| 218259_at | MKL2 | MKL/myocardin-like 2 | 57496 | chr16 | 16p13.12 |
| 218211_s_at | MLPH | Melanophilin | 79083 | chr2 | 2q37.3 |
| 219648_at | MREG | Melanoregulin | 55686 | chr2 | 2q35 |
| 204798_at | MYB | v-myb myeloblastosis viral oncogene homolog (avian) | 4602 | chr6 | 6q22-q23 |
| 214440_at | NAT1 | N-acetyltransferase 1 (arylamine N- | 9 | chr8 | 8p23.1-p21.3 |
| | | acetyltransferase) | | | |
| 204862_s_at | NME3 | non-metastatic cells 3, protein expressed in | 4832 | chr16 | 16q13 |
| 206197_at | NME5 | non-metastatic cells 5, protein expressed in | 8382 | chr5 | 5q31 |
| | | (nucleoside-diphosphate kinase) | | | |
| 202599_s_at | NRIP1 | nuclear receptor interacting protein 1 | 8204 | chr21 | 21q11.2 |
| 222125_s_at | P4HTM | prolyl 4-hydroxylase, transmembrane (endoplasmic | 54681 | chr3 | 3p21.31 |
| | | reticulum) | | | |
| 212148_at | PBX1 | pre-B-cell leukemia homeobox 1 | 5087 | chr1 | 1q23 |
| 217770_at | PIGT | phosphatidylinositol glycan anchor biosynthesis, class T | 51604 | chr20 | 20q12-q13.12 |
| 208615_s_at | PTP4A2 | protein tyrosine phosphatase type IVA, member 2 | 8073 | chr1 | 1p35 |
| 214552_s_at | RABEP1 | rabaptin, RAB GTPase binding effector protein 1 | 9135 | chr17 | 17p13.2 |
| 203749_s_at | RARA | retinoic acid receptor, alpha | 5914 | chr17 | 17q21 |
| 208873_s_at | REEP5 | receptor accessory protein 5 | 7905 | chr5 | 5q22-q23 |
| 212099_at | RHOB | ras homolog gene family, member B | 388 | chr2 | 2p24 |
| 218394_at | ROGDI | rogdi homolog (Drosophila) | 79641 | chr16 | 16p13.3 |
| 201826_s_at | SCCPDH | saccharopine dehydrogenase (putative) | 51097 | chr1 | 1q44 |
| 203071_at | SEMA3B | sema domain, immunoglobulin domain (Ig), short | 7869 | chr3 | 3p21.3 |
| | | basic domain, secreted, (semaphorin) 3B | | | |
| 35666_at | SEMA3F | sema domain, immunoglobulin domain (Ig), short | 6405 | chr3 | 3p21.3 |
| | | basic domain, secreted, (semaphorin) 3F | | | |
| 209443_at | SERPINA5 | serpin peptidase inhibitor, clade A (alpha-1 | 5104 | chr14 | 14q32.1 |
| | | antiproteinase, antitrypsin), member 5 | | | |
| 200718_s_at | SKP1 | S-phase kinase-associated protein 1 | 6500 | chr5 | 5q31 |
| 209681_at | SLC19A2 | solute carrier family 19 (thiamine transporter), | 10560 | chr1 | 1q23.3 |
| | | member 2 | | | |
| 205074_at | SLC22A5 | solute carrier family 22 (organic cation/ | 6584 | chr5 | 5q31 |
| | | carnitine transporter), member 5 | | | |
| 202088_at | SLC39A6 | solute carrier family 39 (zinc transporter), member 6 | 25800 | chr18 | 18q12.2 |
| 205597_at | SLC44A4 | solute carrier family 44, member 4 | 80736 | chr6_qbl_hap2 | 6p21.3 |
| 202752_x_at | SLC7A8 | solute carrier family 7 (cationic amino acid | 23428 | chr14 | 14q11.2 |
| | | transporter, y+ system), member 8 | | | |
| 216092_s_at | SLC7A8 | solute carrier family 7 (cationic amino acid | 23428 | chr14 | 14q11.2 |
| | | transporter, y+ system), member 8 | | | |
| 212956_at | TBC1D9 | TBC1 domain family, member 9 (with GRAM | 23158 | chr4 | 4q31.21 |
| | | domain) | | | |
| 204045_at | TCEAL1 | transcription elongation factor A (SII)-like 1 | 9338 | chrX | Xq22.1 |
| 202371_at | TCEAL4 | transcription elongation factor A (SII)-like 4 | 79921 | chrX | Xq22.2 |
| 205009_at | TFF1 | trefoil factor 1 | 7031 | chr21 | 21q22.3 |
| 204623_at | TFF3 | trefoil factor 3 (intestinal) | 7033 | chr21 | 21q22.3 |
| 212770_at | TLE3 | transducin-like enhancer of split 3 | 7090 | chr15 | 15q22 |
| | | (E(sp1) homolog, Drosophila) | | | |
| 200804_at | TMBIM6 | Transmembrane BAX inhibitor motif containing 6 | 7009 | chr12 | 12q12-q13 |
| 203476_at | TPBG | trophoblast glycoprotein | 7162 | chr6 | 6q14-q15 |
| 217979_at | TSPAN13 | tetraspanin 13 | 27075 | chr7 | 7p21.1 |
| 210652_s_at | TTC39A | tetratricopeptide repeat domain 39A | 22996 | chr1 | 1p32.3 |
| 221765_at | UGCG | UDP-glucose ceramide glucosyltransferase | 7357 | chr9 | 9q31 |
| 218806_s_at | VAV3 | vav 3 guanine nucleotide exchange factor | 10451 | chr1 | 1p13.3 |
| 212637_s_at | WWP1 | WW domain containing E3 ubiquitin protein ligase 1 | 11059 | chr8 | 8q21 |
| 200670_at | XBP1 | X-box binding protein 1 | 7494 | chr22 | 22q12.1\|22q12 |
| 219741_x_at | ZNF552 | zinc finger protein 552 | 79818 | chr19 | 19q13.43 |
| 215304_at | — | — | — | chr15 | — |
| 222275_at | — | — | — | chr5 | — |

Negative Correlation with ESR1

| | | | | | |
|---|---|---|---|---|---|
| 213532_at | ADAM17 | ADAM metallopeptidase domain 17 | 6868 | chr2 | 2p25 |
| 209122_at | ADFP | adipose differentiation-related protein | 123 | chr9 | 9p22.1 |
| 205109_s_at | ARHGEF4 | Rho guanine nucleotide exchange factor (GEF) 4 | 50649 | chr2 | 2q22 |
| 202207_at | ARL4C | ADP-ribosylation factor-like 4C | 10123 | chr2 | 2q37.1 |
| 219497_s_at | BCL11A | B-cell CLL/lymphoma 11A (zinc finger protein) | 53335 | chr2 | 2p16.1 |
| 205548_s_at | BTG3 | BTG family, member 3 | 10950 | chr21 | 21q21.1-q21.2 |
| 219806_s_at | C11orf75 | chromosome 11 open reading frame 75 | 56935 | chr11 | 11q13.3-q23.3 |
| 203256_at | CDH3 | cadherin 3, type 1, P-cadherin (placental) | 1001 | chr16 | 16q22.1 |
| 221676_s_at | CORO1C | coronin, actin binding protein, 1C | 23603 | chr12 | 12q24.1 |
| 203139_at | DAPK1 | death-associated protein kinase 1 | 1612 | chr9 | 9q34.1 |
| 204750_s_at | DSC2 | desmocollin 2 | 1824 | chr18 | 18q12.1 |
| 203693_s_at | E2F3 | E2F transcription factor 3 | 1871 | chr6 | 6p22 |
| 201231_s_at | ENO1 | enolase 1, (alpha) | 2023 | chr1 | 1p36.3-p36.2 |
| 212371_at | FAM152A | family with sequence similarity 152, member A | 51029 | chr1 | 1q44 |
| 212771_at | FAM171A1 | family with sequence similarity 171, member A1 | 221061 | chr10 | 10p13 |
| 213260_at | FOXC1 | forkhead box C1 | 2296 | chr6 | 6p25 |
| 221510_s_at | GLS | Glutaminase | 2744 | chr2 | 2q32-q34 |
| 213170_at | GPX7 | glutathione peroxidase 7 | 2882 | chr1 | 1p32 |
| 200824_at | GSTP1 | glutathione S-transferase pi 1 | 2950 | chr11 | 11q13 |
| 206074_s_at | HMGA1 | high mobility group AT-hook 1 | 3159 | chr6 | 6p21 |
| 202147_s_at | IFRD1 | interferon-related developmental regulator 1 | 3475 | chr7 | 7q22-q31 |
| 206734_at | JRKL | jerky homolog-like (mouse) | 8690 | chr11 | 11q21 |
| 217938_s_at | KCMF1 | potassium channel modulatory factor 1 | 56888 | chr2 | 2p11.2 |
| 204401_at | KCNN4 | potassium intermediate/small conductance | 3783 | chr19 | 19q13.2 |
| | | calcium-activated channel, subfamily N, member 4 | | | |
| 220239_at | KLHL7 | kelch-like 7 (Drosophila) | 55975 | chr7 | 7p15.3 |
| 205569_at | LAMP3 | lysosomal-associated membrane protein 3 | 27074 | chr3 | 3q26.3-q27 |
| 201795_at | LBR | lamin B receptor | 3930 | chr1 | 1q42.1 |
| 213564_x_at | LDHB | lactate dehydrogenase B | 3945 | chr12 | 12p12.2-p12.1 |
| 209205_s_at | LMO4 | LIM domain only 4 | 8543 | chr1 | 1p22.3 |
| 212274_at | LPIN1 | lipin 1 | 23175 | chr2 | 2p25.1 |
| 218684_at | LRRC8D | leucine rich repeat containing 8 family, member D | 55144 | chr1 | 1p22.2 |
| 206571_s_at | MAP4K4 | mitogen-activated protein kinase kinase kinase kinase 4 | 9448 | chr2 | 2q11.2-q12 |
| 203636_at | MID1 | midline 1 (Opitz/BBB syndrome) | 4281 | chrX | Xp22 |
| 201976_s_at | MYO10 | myosin X | 4651 | chr5 | 5p15.1-p14.3 |
| 203315_at | NCK2 | NCK adaptor protein 2 | 8440 | chr2 | 2q12 |
| 203574_at | NFIL3 | nuclear factor, interleukin 3 regulated | 4783 | chr9 | 9q22 |
| 218051_s_at | NT5DC2 | 5′-nucleotidase domain containing 2 | 64943 | chr3 | 3p21.1 |
| 200790_at | ODC1 | ornithine decarboxylase 1 | 4953 | chr2 | 2p25 |
| 209791_at | PADI2 | peptidyl arginine deiminase, type II | 11240 | chr1 | 1p36.13 |
| 201037_at | PFKP | phosphofructokinase, platelet | 5214 | chr10 | 10p15.3-p15.2 |
| 201397_at | PHGDH | phosphoglycerate dehydrogenase | 26227 | chr1 | 1p12 |
| 218236_s_at | PRKD3 | protein kinase D3 | 23683 | chr2 | 2p21 |
| 204061_at | PRKX | protein kinase, X-linked | 5613 | chrX | Xp22.3 |
| 204304_s_at | PROM1 | prominin 1 | 8842 | chr4 | 4p15.32 |
| 200039_s_at | PSMB2 | proteasome (prosome, macropain) subunit, beta type, 2 | 5690 | chr1 | 1p34.2 |
| 212265_at | QKI | quaking homolog, KH domain RNA binding | 9444 | chr6 | 6q26\|6q26-q27 |
| | | (mouse) | | | |
| 213923_at | RAP2B | RAP2B, member of RAS oncogene family | 5912 | chr3 | 3q25.2 |
| 221872_at | RARRES1 | retinoic acid receptor responder (tazarotene induced) 1 | 5918 | chr3 | 3q25.32-q25.33 |
| 218497_s_at | RNASEH1 | ribonuclease H1 | 246243 | chr2 | 2p25 |
| 213113_s_at | SLC43A3 | solute carrier family 43, member 3 | 29015 | chr11 | 11q11 |
| 210959_s_at | SRD5A1 | steroid-5-alpha-reductase, alpha polypeptide 1 | 6715 | chr5 | 5p15 |
| | | (3-oxo-5 alpha-steroid delta 4-dehydrogenase alpha 1) | | | |
| 202200_s_at | SRPK1 | SFRS protein kinase 1 | 6732 | chr6 | 6p21.3-p21.2 |
| 202951_at | STK38 | serine/threonine kinase 38 | 11329 | chr6 | 6p21 |
| 221016_s_at | TCF7L1 | transcription factor 7-like 1 (T-cell specific, HMG-box) | 83439 | chr2 | 2p11.2 |
| 211967_at | TMEM123 | Transmembrane protein 123 | 114908 | chr11 | 11q22.1 |
| 202342_s_at | TRIM2 | tripartite motif-containing 2 | 23321 | chr4 | 4q31.3 |
| 202504_at | TRIM29 | tripartite motif-containing 29 | 23650 | chr11 | 11q22-q23 |
| 208627_s_at | YBX1 | Y box binding protein 1 | 4904 | chr7 /// chr9 | 1p34 |
| 221203_s_at | YEATS2 | YEATS domain containing 2 | 55689 | chr3 | 3q27.1 |

TABLE 3
Summary of available samples and the total number of microarrays analyzed.

| | Sample Cohorts Evaluated | | | | | |
|---|---|---|---|---|---|---|
| | | 1^{st} | 2^{nd} | 1^{st} | 2^{nd} | Chemo- |
| | Discovery | Tamoxifen | Tamoxifen | Untreated | Untreated | Endocrine |
| Dates samples | 2000-2007 | 1987-1997 | 1978-2002 | 1980-1995 | 1980-1998 | 2000-2006 |
| collected | | | | | | |
| Insufficient RNA | 80 | ~60 | 1 | 97 | 104 | |
| amount or quality | | | | | | |
| Microarrays | 460 | 245 | 309 | 286 | 198 | |
| evaluated | | | | | | |
| Microarrays failed | 23 | 4 | 7 | 0 | 2 | 1* |
| ER-negative cases | NA | 9 | 0 | 77 | 63 | |
| DRFS unavailable | NA | 7 | 4 | 1 | 0 | 9* |
| or <6 months | | | | | | |
| Total microarrays | 437 | 225 | 298 | 208 | 133 | 122* ²⁰ |
| analyzed | | | | | | |

*A published subset of our discovery cohort, from whom we excluded one microarray that failed our quality control, and nine patients who had only received endocrine therapy as palliative treatment (N = 7), refused adjuvant endocrine therapy (N = 1), or were lost to follow up (N = 1).

Calculation of Sensitivity to Endocrine Treatment Index.

To quantify the expression of the 165 reporter genes in new samples, the inventors first developed a gene-expression-based ER reporter index (ERI). Let X_{N} and X_{P} be the mean expression value of the 59 negatively-correlated and 106 positively correlated genes with ESR1 in a given sample. Then an endocrine pathway index is defined as EI=X_{N}+f (X_{P}−X_{N}), where f is a constant between 0 and 1. Typical values include 0.64, which is the fraction of positively associated genes (106/165) or 0.5. The most typical value is f=0.5. In ER-negative tumors, expression of both the positively and negatively ESR1 correlated genes is low and therefore EI is small. In ER-positive tumors, expression the positively correlated genes will be greater than that of the negatively correlated genes and therefore the index takes on positive values.

The EI is further transformed to obtain less extreme values that better conform to a normal distribution, which helps in subsequent analysis for establishing the cutpoints to define response groups. The final form of the genomic index of sensitivity to endocrine therapy (SET) is calculated from EI as follows: SET=max {0, A (EI+B)^{P}_{}}. Constant B is an offset determined to produce positive values for the index, A is an arbitrary scale constant and exponent p was determined through an unconditional Box-Cox power transformation for normality. The most typical values of these constants are A=10, B=−9.48 and p=1.24. The above formulation for SET means that SET is zero-truncated, i.e. if the result of the formula is negative it is set equal to zero.

Cutoff points were established to classify the sensitivity to endocrine therapy index to low, intermediate, or high. Cutoff points of the SET index values were determined from a subset of the evaluation dataset of treated patients (evaluation cohort of patients treated with adjuvant tamoxifen, n=245). Among the 245 samples, a total of 20 cases were excluded from this analysis because of patients were ER-negative, or did not have follow up information, or events occurred within 5 months after surgery, or they did not pass microarray QC. The subset of 225 cases was used to define the 2 cutoff points. A Cox regression model was fit to predict DRFS in relation to the trichotomous SET indicator variable using different thresholds. Thresholds that resulted in maximum or near maximum log-profile likelihood for this model were selected as most informative cut points for predicting DRFS (Tableman and Kim, 2004). The same thresholds were maintained for all subsequent analyses of the treated and untreated patients. Typical values of these thresholds were 3.86 and 4.08.

### Example 2

### Correlation Between ER mRNA Expression Levels and ER Status

Intensity values of ESR1 (ER) gene expression from microarray experiments were compared to the results from standard IHC and enzyme immunoassays in 82 FNA samples (MDACC). The Affymetrix U133A GeneChip™ has six probe sets that recognize ESR1 mRNA at different sequence locations. A comparison of the different probe sets using the 82 FNA dataset is presented in Table 4. All the ESR1 probe sets showed high correlation with ER status determined by immunohistochemistry (Kruskal-Wallis test, p<0.0001). The probe set 205225_had the highest mean, median, and range of expression and was most correlated with ER status (Spearman's correlation, R=0.85, Table 4).

TABLE 4
The mean, median, and range of expression of the six probe
sets that identify ERα gene (ESR1) are compared using the
results from 82 FNA samples. Expression of each ESR1
probe set is correlated to ER status (positive, low, or
negative) and to the expression of the ESR1 205225_probe
set (R values, Spearmans rank correlation test).

| | Probe Set | | | | |
|---|---|---|---|---|---|
| | | | | I. SPEARMAN | |
| | Signal Intensity | | | CORRELATION WITH | |
| ER ESR1 | Mean | Median | Range | ER Status | 205225_{—} |
| 205225_{—} | 1633 | 912 | 6802 | 0.85 | 1.00 |
| 215552_{—} | 192 | 136 | 671 | 0.81 | 0.86 |
| 217190_{—} | 152 | 122 | 429 | 0.72 | 0.84 |
| 211233_{—} | 234 | 178 | 663 | 0.71 | 0.88 |
| 211235_{—} | 189 | 139 | 674 | 0.69 | 0.88 |
| 211234_{—} | 236 | 209 | 462 | 0.64 | 0.83 |

### Example 3

### Establishing Classes of SET Index and Independence of SET Index from Genomic Performance of Predictors in Multivariate Survival Analyses

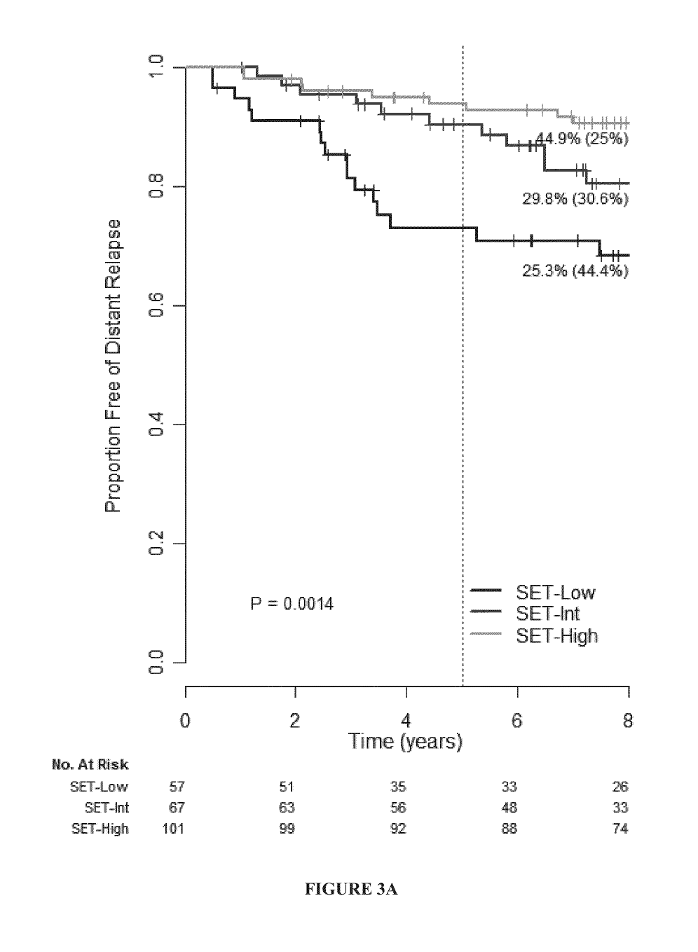

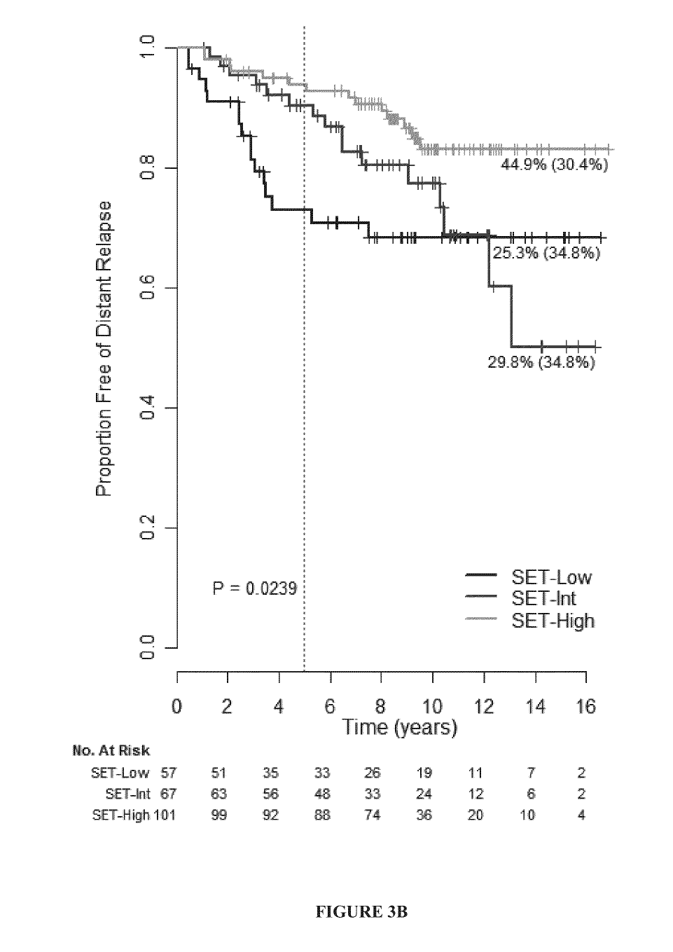

Optimal thresholds to determine the three classes of SET were chosen with a usable subset of the first validation cohort consisting of 225 patients to maximize the predictability of the trichotomous SET index in a multivariate Cox model. Two cut points (corresponding to index values 3.86 and 4.08) were chosen to maximize the association of the trichotomous SET index with distant relapse events or death that occurred within the first 8 years of follow up (FIG. 3A). This trichotomous gene-expression-based SET index was evaluated in a multivariate Cox model in relation to its association with DRFS. Covariates included in the Cox analysis were, in addition to the trichotomous SET index, age at diagnosis, nodal status at surgery, tumor stage (revised American Joint Committee on Cancer (AJCC) staging system), and tumor histologic grade. The SET index, evaluated as hazard ratio between Intermediate to Low, and High to Low, was a significant predictor of relapse after adjuvant tamoxifen treatment (Table 5 below), whereas the effect of almost all other clinical covariates was not statistically significant (Table 5 below). Among the clinical covariates, only tumor size (T-stage II or III versus stage I) had a borderline statistically insignificant association with DRFS (p=0.04). Therefore the SET index was independently predictive of benefit from adjuvant tamoxifen therapy in multivariate analyses accounting for the contributions of other clinical variables.

TABLE 5
Multivariate Cox analysis of SET index to predict DRFS
in patients with ER-positive breast cancer. Treated
patients (n = 209, evaluation cohort with complete
information) received adjuvant tamoxifen for 5 years.

| | | | P |
|---|---|---|---|
| Effect | | HR (95% CI) | value |
| Age | >50 versus ≦50 | 0.98 (0.94 to 1.02) | 0.40 |
| Nodal Status | Positive versus negative | 1.71 (0.79 to 3.70) | 0.18 |
| T Stage | II or III versus I | 2.32 (1.03 to 5.23) | 0.04 |
| Histologic Grade | 3 versus 2 or 1 | 0.81 (0.35 to 1.89) | 0.63 |
| ESR1 Expression | Continuous | 0.93 (0.69 to 1.25) | 0.62 |
| SET Index | Continuous | 0.65 (0.46 to 0.91) | 0.01 |

### Example 4

### Analysis of SET Index Classes in Patients Treated with Adjuvant Tamoxifen

The three classes of predicted sensitivity to endocrine therapy (Low, Intermediate, and High sensitivity) were evaluated for correlation with DRFS in an independent non-overlapping cohort of 310 patients (see Table 1). A subset of 269 patients with complete treatment information was selected for the multivariate Cox regression analysis of which 239 patients had complete information on all variables for the analyses. The results are summarized in Table 6. The SET class was significantly independently predictive of DRFS in the validation cohort as well (p=0.033).

TABLE 6
Multivariate Cox analysis of SET classes to predict DRFS
in an independent cohort of patients with ER-positive breast
cancer. Treated patients (n = 269, validation cohort
with complete information) received adjuvant tamoxifen
for 5 years. * Data of 230 patients were available to perform
the complete multivariate analyses.

| | Hazard | | |
|---|---|---|---|
| Factor | Ratio | 95% CI | P value |
| Age (>50 vs ≦50) | 5.12 | 0.70-37.6 | 0.108 |
| Nodal Status (pos vs neg) | 2.83 | 1.49-5.35 | 0.001 |
| T Stage (II or III vs I) | 1.91 | 0.92-3.97 | 0.082 |
| Histologic Grade (3 vs 1 or 2) | 1.16 | 0.59-2.28 | 0.673 |
| Allred Score ER IHC (≦6 vs 7 or 8) | 1.20 | 0.66-2.21 | 0.549 |
| SET Class (Low or Intermediate vs | 3.64 | 1.11-11.95 | 0.033 |
| High) | | | |

* Sixty eight cases were removed from the multivariate analysis of the tamoxifen validation cohort due to partially missing data. Likelihood ratio test for the addition of SET Class was 6.57 on one degree of freedom, p = 0.010. The Hazard Ratio is a measure of the risk of distant relapse or death; vs., versus; ER IHC, immunohistochemistry for estrogen receptor.

Kaplan-Meier curves of DRFS were estimated for the 3 SET classes over the entire period of follow-up of the patients, first, in the evaluation cohort and then, in the independent non-overlapping validation cohort. In the evaluation cohort, which was also used to establish the cut points thresholds, the three groups of High, Intermediate and Low sensitivity showed statistically significant separation of DRFS (FIG. 3, p=0.0014 over 8 years, and p=0.024 over 16 years follow-up of patients).

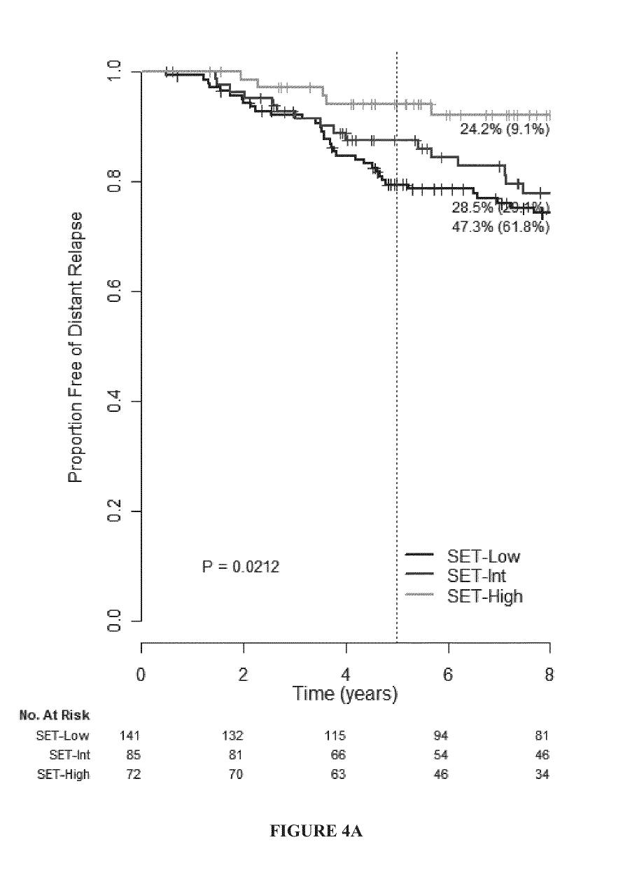

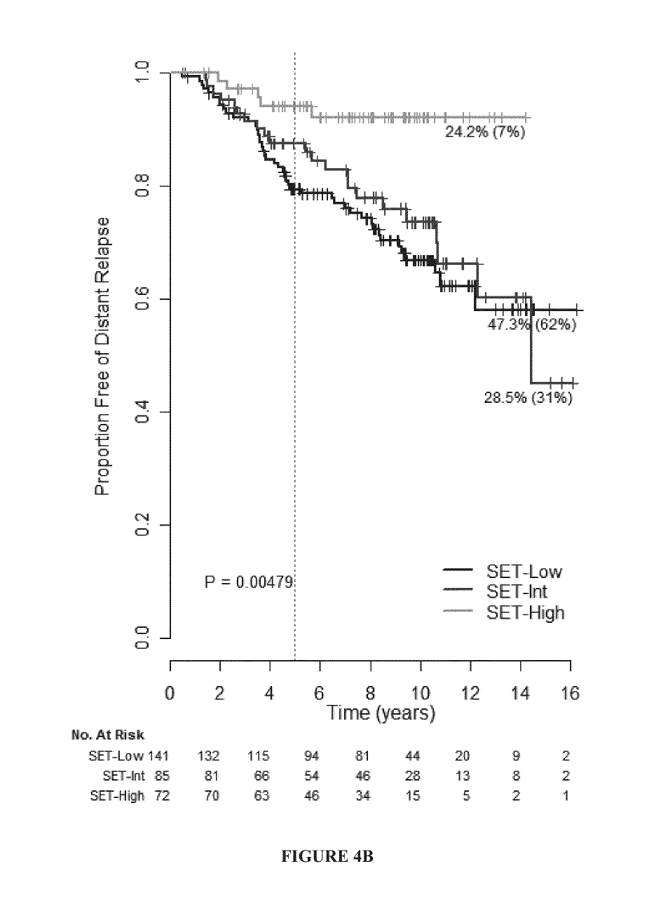

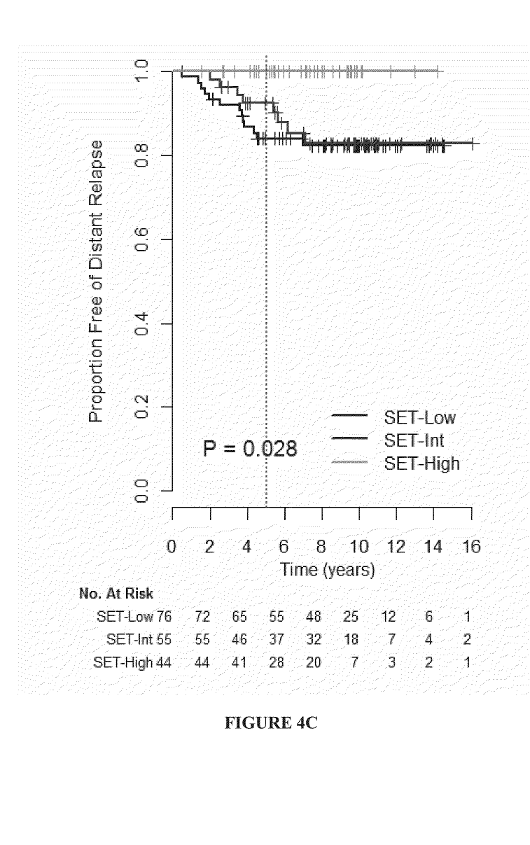

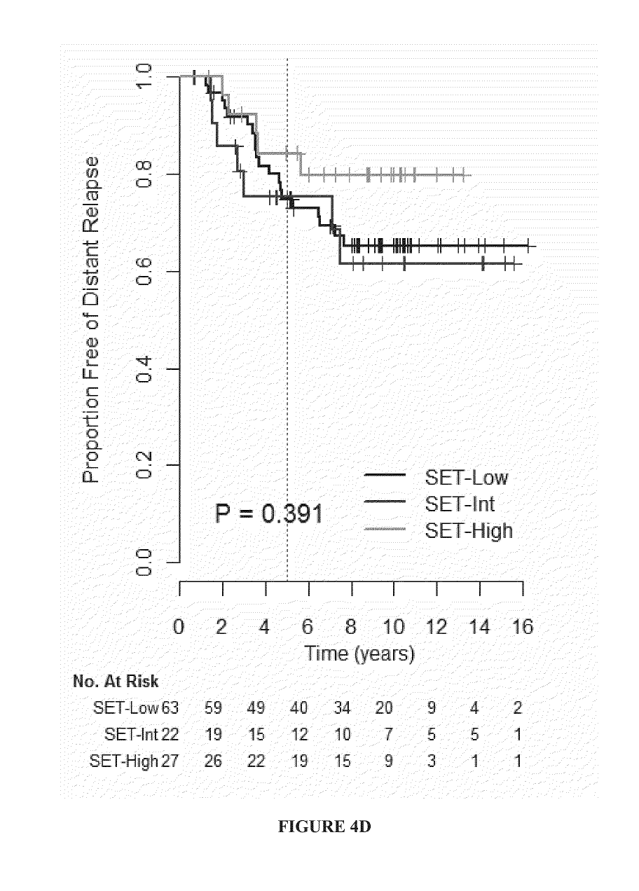

To provide independent validation of these results, a subsequent analysis of DRFS was performed with a treated patient cohort (n=298 patients of 310 total) by using the previously established cutoff points for the three classes. Patients with high endocrine sensitivity (High SET index) had sustained benefit from adjuvant tamoxifen (FIG. 4). Patients with low SET index values derived minimal benefit from adjuvant tamoxifen, irrespective of nodal status. The SET index was developed to represent and measure broad transcriptional activity related to ER within breast cancer samples in order to address a hypothesis that such measure is strongly associated with intrinsic sensitivity to adjuvant endocrine therapy. This study demonstrates and confirms that SET is predictive of distant relapse risk in tamoxifen-treated patients (Table 6, FIGS. 3 and 4). However, lymph node status remained independently prognostic in the tamoxifen-treated patients (FIGS. 4C and 4D), such that node-negative patients with high SET had excellent DRFS from adjuvant endocrine therapy alone (FIG. 4C), whereas node-positive patients with high SET index remained at risk for relapse (FIG. 4D). Therefore, it is important to consider whether chemotherapy should be recommended for patients with node-positive and ER-positive breast cancer, or whether a predictive test for endocrine sensitivity would identify patients with either excellent survival without chemotherapy or for whom added chemotherapy is futile. Albain et al. (2010) have reported that all subgroups of patients with node-positive ER-positive breast cancer remain at significant risk even if predicted to have good prognosis with adjuvant tamoxifen (low recurrence score), or if they also receive adjuvant chemotherapy. In that study, recurrence score identified a subset where chemotherapy offered no relative benefit, but also failed to identify a subset with excellent survival (absolute benefit) from either treatment arm.

### Example 5

### Analysis of SET Index Classes in Untreated Patients—to Demonstrate that SET Index is Independent of Prognosis

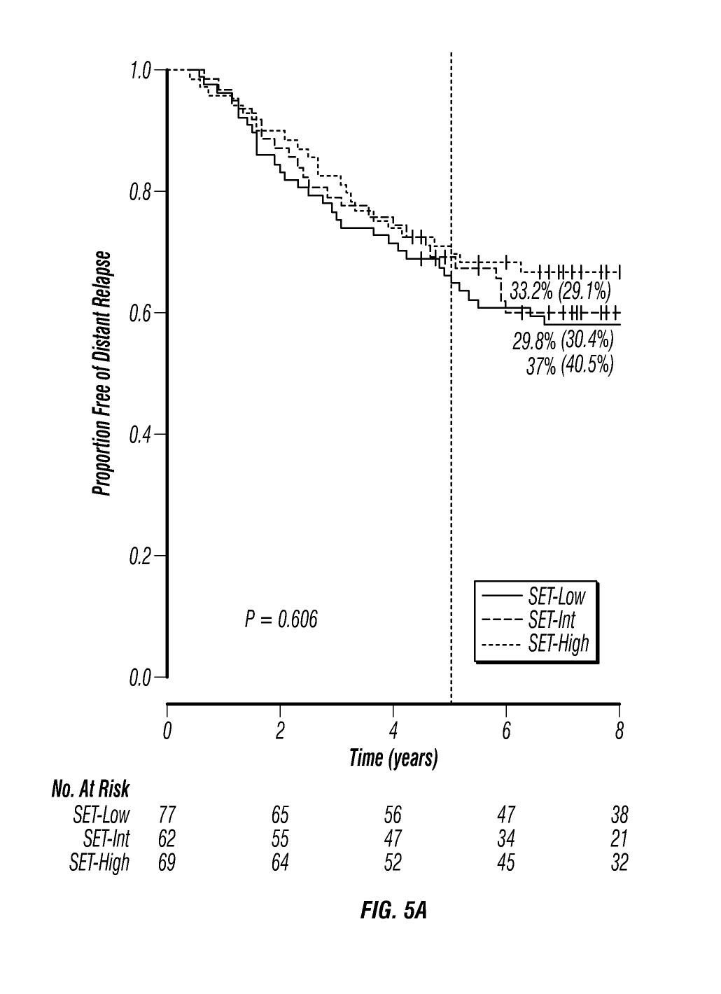

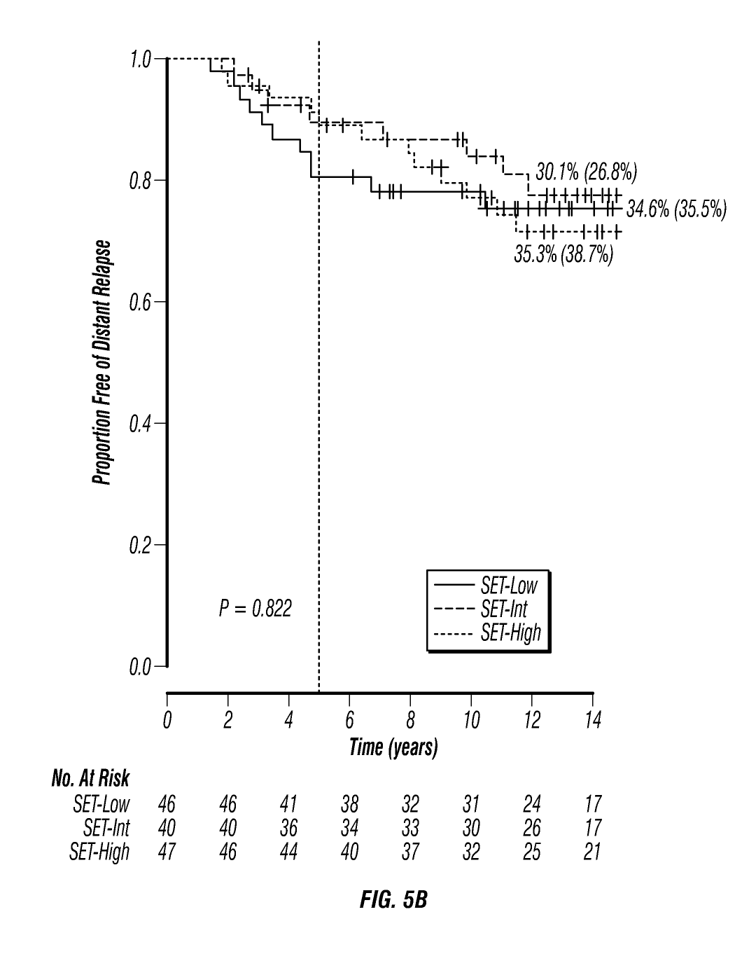

To address the possibility that observed differences in DRFS could be due to indolent prognosis, rather than benefit from adjuvant tamoxifen, the same SET index classes with the established cut-points were evaluated as potential prognostic factors of DRFS in patients who did not receive any systemic therapy. Two independent patient cohorts, who had node-negative breast cancer, were employed for this analysis: (i) 208 ER-positive patients marked as VDX in Tables 1 and 2, and (ii) 133 ER-positive patients marked TRANS in Tables 1 and 2. FIG. 5 shows distant relapse events in both groups of patients classified by High, Intermediate, and Low SET index values. As the Figure indicates, the separation of survival between SET classes is poor and statistically insignificant (p=0.606 and p=0.822, respectively in the two independent cohorts). Thus, the SET index and its classes are independent of prognosis after surgery and are highly correlated with survival as a benefit of tamoxifen therapy as demonstrated in Example 4.

### Example 6

### Association of SET Index with DRFS after Adjuvant Chemo-Endocrine Therapy

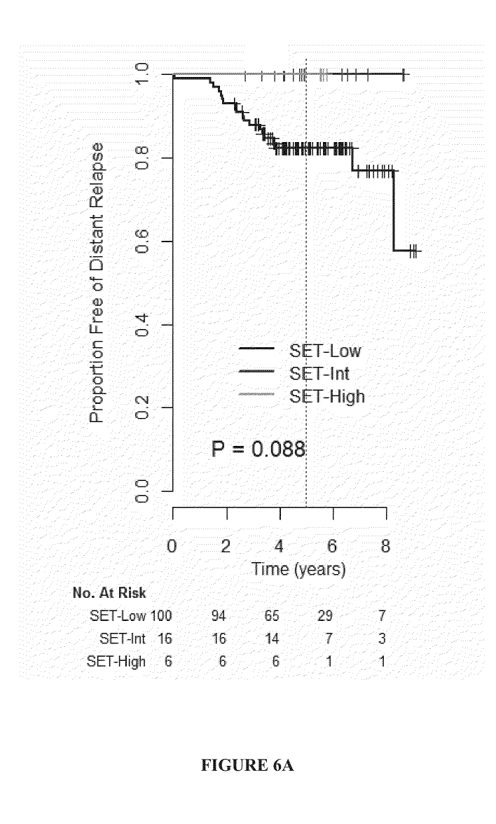

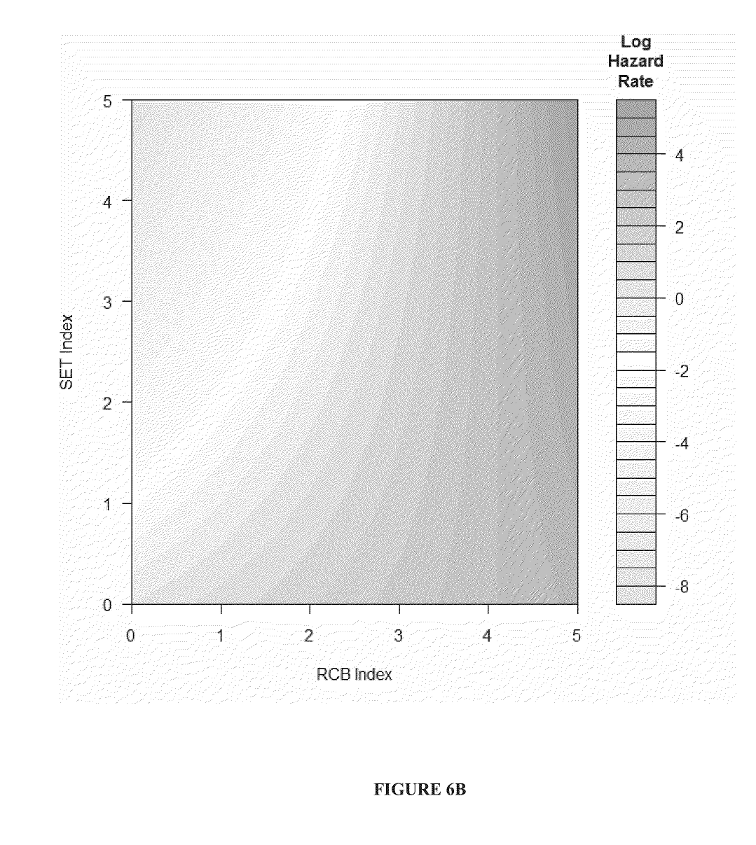

Patients with high or intermediate SET index had similar frequency of clinical node-positive status at presentation (12/22 versus 68/100), and pathologic response from neoadjuvant chemotherapy (3/22 versus 5/100 pCR, 6/22 versus 35/100 pCR/RCB-I) compared to low SET (Chi-square tests not significant). However, the point estimates of DRFS for high or intermediate, and low SET index categories at 5 years of follow up were 100% (95% CI 100 to 100) and 82.4% (95% CI 75.1 to 90.4), respectively (FIG. 6A). Indeed, response from chemotherapy measured by the residual cancer burden (RCB) index, (Symmans et al., 2007) and by the SET index were each independently predictive of distant relapse risk, and their interaction term was also borderline significant (Table 7). To illustrate this interaction (FIG. 6B), elevated endocrine sensitivity (SET index) appears to be associated with reduced relapse risk when there is less than extensive RCB after chemotherapy, and particularly when RCB is low.

TABLE 7
Multivariate Cox analysis of SET classes in an independent cohort
of patients with ER-positive breast cancer (n = 122) treated
with neoadjuvant chemotherapy and adjuvant endocrine therapy.
T/FAC Chemotherapy Followed By Tamoxifen
and/or Aromatase Inhibition (N = 122)**

| | Hazard | | |
|---|---|---|---|
| Factor | Ratio | 95% CI | P value |
| Residual Cancer Burden (continuous) | 2.07 | 1.20-3.60 | 0.01 |
| SET index (continuous) | 0.19 | 0.05-0.69 | 0.01 |
| Interaction Term (RCBxSET) | 1.49 | 0.99-2.24 | 0.05 |

**Likelihood ratio test for the addition of SET index and interaction term was 8.45 on 2 degrees of freedom, p = 0.015. The Hazard Ratio is a measure of the risk of distant relapse or death; vs., versus; ER IHC, immunohistochemistry for estrogen receptor.

In this Example, the SET index is analyzed in a population with clinical Stage II-III ER-positive HER2-negative breast cancer who had been selected for neoadjuvant chemotherapy followed by current endocrine therapy. These were not from a randomized population, and so relative benefit from chemotherapy cannot be evaluated according to SET index. However, response to the chemotherapy as assessed by the extent of residual disease through the RCB index and the endocrine sensitivity (SET index) could both be evaluated as predictors of distant relapse risk after the combined therapy. High or intermediate SET index were not associated with pathologic response, but imparted excellent 5-year survival (FIG. 6A). Furthermore, SET index was predictive of relapse risk independently from chemotherapy response (Table 7) and had an apparent synergistic interaction with RCB, with a stronger predictive association between increasing SET values and lower risk of death or distant relapse when there is less residual disease after neoadjuvant chemotherapy (FIG. 6B). This suggests that partial benefit from chemotherapy can further improve the survival of patients receiving endocrine therapy for higher risk intrinsically endocrine-sensitive disease, and further supports our interpretation of SET index as an independent predictor of benefit from subsequent adjuvant endocrine therapy.

In the above Examples, approximately 25% of patients with ER-positive node-negative breast cancer had high SET index values and excellent survival from 5 years of endocrine therapy alone. Another 30% of patients with intermediate SET index values might benefit more from chemo-endocrine or prolonged and different endocrine therapy, but 25% to 50% patients with low SET index might be advised to consider chemo-endocrine therapy. Approximately 20% of patients with clinical stage II-III disease had high or intermediate SET index and excellent 5-year DRFS that was independent of their chemotherapy response, but attributable to sequential benefits from chemo-endocrine therapy.

### REFERENCES

The following references, to the extent that they provide exemplary procedural or other details supplementary to those set forth herein, are specifically incorporated herein by reference.
Albain et al., Lancet. Oncol., 11:55-65, 2010.
Ayers et al., J. Clin. Oncol., 22:2284-2293, 2004.
Blankenstein et al., Clin. Chim. Acta, 165L189-195, 1987.
Bonneterre et al., J. Clin. Oncol., 18:3748-57, 2000.
Bryant and Wolmark, N. Engl. J. Med., 349(19):1855-1857, 2003.
Burstein, N. Engl. J. Med., 349(19):1857-1859, 2003.
Buzdar, Semin. Oncol., 28:291-304, 2001.
Esteva et al., Clin. Cancer Res., 11:3315-9, 2005.
Gong et al. Lancet. Oncol., 8(3):203-11, 2007.
Gong et al., Cancer, 102:34-40, 2004.
Goss et al., N. Engl. J. Med., 349(19):1793-1802, 2003.
Gruvberger-Saal et al., Mol. Cancer Ther., 3:161-168, 2004.
Gruvberger et al., Cancer Res., 61:5979-5984, 2001.
Harvey et al., J. Clin. Oncol., 17:1474-1481, 1999.
Hess et al., Breast Cancer Res. Treat., 78:105-118, 2003.
Howell and Dowsett, Breast Cancer Res., 6:269-274, 2004.
Howell et al., Lancet., 365(9453):60-62, 2005.
Jansen et al., J. Clin. Oncol., 23:732-740, 2005.
Kendall and Gibbons, In: Rank Correlation Methods, NY, Oxford University Press, 1990.
Konecny et al., J. Natl. Cancer Inst., 95:142-153, 2003.
Kun et al., Hum. Mol. Genet., 12:3245-3258, 2003.
Lacroix et al., Breast Cancer Res. Treat., 67:263-271, 2001.
Loi et al., Proc. Am. Soc. Clin. Oncol., Abstract #509, 2005
Ma et al., Cancer Cell, 5:607-616, 2004.
Mouridsen et al., J. Clin. Oncol., 19:2596-2606, 2001.
Paik et al., N. Engl. J. Med., 351:2817-2826, 2004.
Paik et al., Proc. Am. Soc. Clin. Oncol., Abstract #510, 2005.
Pepe et al., Biometrics, 59:133-142, 2003.
Perou et al., Nature, 406:747-752, 2000.
Pusztai et al., Clinical Cancer Res., 9:2406-2415, 2003.
Ransohoff, Nat. Rev. Cancer, 4:309-314, 2004.
Ransohoff, Nat. Rev. Cancer, 5:142-149, 2005.
Regitnig et al., Virchows Arch., 441:328-34, 2002.
Rhodes et al., J. Clin. Pathol., 53:125-130, 2000.
Rhodes, Am. J. Surg. Pathol., 27(9):1284-1285, 2003.
Rudiger et al., Am. J. Surg. Pathol., 26:873-882, 2002.
Sorlie et al., Proc. Natl. Acad. Sci. USA, 98:10869-10874, 2001.
Sotiriou et al, J. Natl. Cancer Inst., 98:262-72, 2006
Symmans et al., Cancer, 97:2960-2971, 2003.
Symmans et al., J. Clin. Pathol, 25:4414-4422, 2007.
Tableman and Kim, In: Survival Analysis Using S: Analysis of Time-to-Event Data, F L:
Chapman & Hall/CRC; 2004.
Taylor et al., Hum. Pathol., 25:263-270, 1994.
Therneau and Grambsch, In: Modeling Survival Data: Extending the Cox Model, NY, Springer-Verlag; 2000.
Thurlimann et al., N. Engl. J. Med., 353(26):2747-2757, 2005.
van't Veer et al., Nature, 415:530-536, 2002.

1. A method of assessing cancer patient sensitivity to treatment comprising the step of calculating a sensitivity to endocrine therapy (SET) index score for the patient's tumor based on expression in a tumor sample from the patient of one or more genes selected from Table 2.

2. The method of claim 1, further comprising selecting a treatment based on the SET index.

3. The method of claim 1, wherein the ER-related genes comprise 25 or more ER related genes of Table 2.

4-5. (canceled)

6. The method of claim 4, wherein the ER-related genes comprise 165 ER related genes of Table 2.

7. The method of claim 1, wherein the SET index includes covariates of tumor size, nodal status, grade, and age.

8. The method of claim 1, wherein the SET index includes evaluation of overall survival (OS).

9. The method of claim 8, wherein the SET index includes evaluation of distant relapse-free survival (DRFS).

10. The method of claim 1, wherein the treatment is a combination of one or more cancer therapy.

11. The method of claim 1, wherein the treatment is hormonal therapy, chemotherapy, or both.

12. The method of claim 11, wherein the hormonal therapy is tamoxifen therapy, aromatase inhibitor therapy, or SERM therapy.

13-14. (canceled)

15. The method of claim 1, wherein the patients are diagnosed with early or late-stage cancer.

16-19. (canceled)

20. The method of claim 11, further comprising selecting a class or individual hormonal therapy.

21. The method of claim 20, wherein the hormonal therapy is tamoxifen therapy, aromatase inhibitor therapy, or SERM therapy.

22. The method of claim 1, further comprising identifying a patient that will benefit from an extended duration of therapy.

23-28. (canceled)

29. The method of claim 1, wherein the SET index includes a metric indicative of ER status of all or part of the reference tumor samples.

30-34. (canceled)

35. The method of claim 1, wherein the expression data of the one or more genes from the patent's tumor sample is normalized to a digital standard.

36. The method of claim 35, wherein the digital standard is a gene expression profile from a reference sample.

37. A kit to determine ER status of cancer comprising:
(a) reagents for determining expression levels of one or more ER related genes selected from Table 2 in a sample; and
(b) an algorithm and software encoding the algorithm for calculating an ER reporter index from the expression ER related genes in a sample to determine the sensitivity of the patient to hormonal therapy.

38-41. (canceled)

42. A method for analyzing ER transcriptional activity comprising;
(a) providing an array of locations containing nucleic acid hybridization sites;
(b) hybridizing the array of locations with a nucleic acid sample obtained from a sample;
(c) scanning the nucleic acid hybridization site in each location on the array to obtain signals from the hybridization sites corresponding to ER related genes analyzed, wherein the hybridization sites provide ER related gene expression data for genes selected from Table 2;
(d) converting the ER related gene expression data into digital data; and
(e) utilizing the digital data to make assessments as compared to a reporter index, wherein the assessments are used to determine hormonal sensitivity of a patient's cancer.
